(19) United States
(12) Patent Application Publication
(10) Pub. No.: US 2004/0234505 A1
(43) Pub. Date: Nov. 25, 2004

(54) Polynucleotide constructs and uses thereof

(76) Inventors: Stuart Naylor, The Oxford Science Park (GB); Susan Mary Kingsman, The Oxford Science Park (GB); Katie Binley, The Oxford Science Park (GB)

Correspondence Address: FROMMER LAWRENCE & HAUG, 745 FIFTH AVENUE- 10TH FL., NEW YORK, NY, 10151, US

(21) Appl. No.: 10/810,262

(22) Filed: Mar. 26, 2004

Related U.S. Application Data
(63) Continuation-in-part of application No. 09/787,562, filed on Jul. 6, 2001.
Application No. 09/787,562 is a 371 of international application No. PCT/GB99/03181, filed on Sep. 22, 1999.

(30) Foreign Application Priority Data
Sep. 23, 1998 (WO) PCT/GB98/02885
Jan. 28, 1999 (GB) 9901906.9
Feb. 16, 1999 (GB) 9903538.8

(51) Int. Cl. A61K 48/00

(52) U.S. Cl. 424/93.2; 514/44

(57) ABSTRACT

Provided is a method for treating ocular neovascularization by delivering a vector, preferably a viral vector, to target cells in the eye of a subject. The vector contains a a polynucleotide sequence encoding an angiostatic gene product, under the control of a promoter sequence. The angiostatic gene product is expressed in the target cells, thereby treating the ocular neovascularization in the subject.

### CROSS REFERENCE TO RELATED APPLICATIONS

This application is a continuation-in-part of U.S. application Ser. No. 09/787,562, filed on Jul. 6, 2001, which is a National Phase application under 35 U.S.C. §371 of International application No. PCT/GB99/03181, filed on Sep. 22, 1999, which claims priority to International application No. PCT/GB98/02885, filed on Sep. 23, 1998, to UK application No. 9901906.9, filed on Jan. 28, 1999 and to UK application No. 9903538.8, filed on Feb. 16, 1999.

This application makes reference to U.S. Pat. No. 6,265,390, filed on Feb. 22, 1999, which is a divisional of U.S. application Ser. No. 08/693,174, filed on Dec. 12, 1996, now U.S. Pat. No. 5,942,434, which is a National Phase application under 35 U.S.C. §371 of International application No. PCT/GB95/00322, filed on Feb. 15, 1995 and claiming priority to UK application No. 9402857, filed on Feb. 15, 1994. This application also makes reference to U.S. Pat. No. 6,312,682, filed on Dec. 28, 1998, to U.S. Pat. No. 6,312,683, filed on Jan. 27, 1999, and to U.S. application Ser. Nos. 10/066,218, filed on Feb. 1, 2002, and Ser. No. 10/008,610, filed on Nov. 8, 2001.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to polynucleotide constructs or vectors for delivery of polynucleotide sequences encoding angiostatic gene products to target cells in or associated with a hypoxic environment. The polynucleotide constructs or vectors are useful for treatment of diseases associated with hypoxia, and also find use in the identification of novel therapeutic targets within the hypoxia-signaling pathway. In a preferred embodiment of the invention, the polynucleotide constructs or vectors comprise hypoxia response elements (HREs) and/or polynucleotide sequences encoding angiostatic gene products.

### BACKGROUND OF THE INVENTION

Hypoxia (low oxygen) is a defining physiological feature of a number of diseases, including cancer, cardiovascular disease, and ocular diseases such as retinopathies. Hypoxia plays an active role in the pathology of these diseases through its impact on gene expression, thereby making the hypoxia-signalling pathway a key target for the development of novel molecular therapies and target validation strategies.

Gene therapy, the treatment or prevention of disease by gene transfer, is one of a number of approaches being used to treat a range of diseases in animals, particularly in humans. Long-term uncontrolled gene expression is often undesirable for many gene therapy applications. Using a physiologically regulated promoter such as a hypoxia-responsive promoter, it is possible to specifically target gene expression to a particular region of diseased tissue that is known to be hypoxic or to have elevated levels of HIF-1 transcription factor (the key mediator of the hypoxia signalling cascade). In addition, therapeutic gene expression will cease if or when the disease resolves and/or the hypoxia/HIF-1 levels return to normal.

There are numerous retinopathies where ischaemia is known to be of central importance to the disease pathology. These include diabetic retinopathy, age-related macular degeneration, retinopathy of prematurity, central vein occlusion, branch vein occlusion, and ocular ischaemia syndrome. By far the most clinically important of these diseases are age-related macular degeneration (AMD) and diabetic retinopathy (PDR)

Retinal and choroidal neovascularisation are the most common causes of severe visual loss in developed countries. Clinically, ocular angiogenesis is of enormous significance because retinal neovascularisation resulting from diabetic retinopathy is the most common cause of new blindness in young patients, and choroidal neovascularisation (CNV) resulting from AMD is the most common cause of vision loss in individuals over the age of fifty-five. In the United States more than 6 million individuals have a severe visual impairment related to AMD, this represents about 60% of all patients with reported visual loss. In fact, as many as 1 in 4 people over the age of 65 have some form of macular degeneration.

AMD exists in two forms, dry and wet. The dry form is characterised by the presence of lipid-containing deposits, called drusen, which form beneath the retinal pigment epithelium (RPE) in the macular region. Dry AMD accounts for about 90% of all cases, but its impact on vision is less devastating than the wet form. However, individuals with the dry form of AMD may eventually go on to develop the wet-form of AMD.

Wet AMD is characterised by CNV, a condition in which abnormal blood vessel growth in the vascular choroids eventually breaks through the basement membrane (Bruch's membrane) of the RPE and invades the outer retina. The blood vessels are abnormal and often leak blood and fluid that damage the photoreceptor cells. Approximately 10% of all AMD cases are of the wet form, however these cases account for the majority of severe vision impairment in AMD. Wet AMD tends to progress rapidly and cause severe damage to central vision.

The retina is supplied by two vascular beds. Retinal vessels supply the inner two-thirds, and to accommodate visual function, the outer retina is completely avascular and receives oxygen and nutrients from the choroidal circulation. To enhance transport, there is a large collection of choroidal capillaries beneath the retina, referred to as the choriocapillaris. Plasma leaks from the choriocapillaris and pools beneath the RPE, which has tight junctions and several transport systems—this constitutes the outer blood-retinal barrier. The retinal vascular endothelial cells have tight junctions and constitute the inner blood-retinal barrier.

The pathogenesis of retinal neovascularisation is better understood than the pathogenesis of CNV. Numerous clinical and experimental studies have indicated that ischaemia (or hypoxia) plays a central role in the development of retinal neovascularisation and occurs by the occlusion of retinal vessels. Vascular endothelial growth factor (VEGF) is a major stimulatory factor for retinal neovascularisation. It is unlikely to be the only stimulatory factor, since insulin-like growth factor I seems likely to participate (Smith LE et al 1997 Science) but it is nevertheless the key factor involved. VEGF is upregulated by hypoxia and its levels are increased in the retina and vitreous of patients or laboratory animals with ischaemic retinopathies (reviewed in Campochiaro P 1999, Molec. Vision). Also, increased expression of VEGF in retinal photoreceptors or rhodopsin/VEGF transgenic mice stimulates neovascularisation in the retina and VEGF antagonists inhibit retinal or indeed iris neovascularisation in animal models.

There is more circumstantial evidence that VEGF is a stimulatory factor for CNV. In animal models of laser-induced CNV, increases in VEGF mRNA are seen in RPE-like cells, choroidal vascular endothelial cells and fibroblast-like cells in the lesions (Yi X et al 1997, Graefes Arch Clin Exp Oph. Husain D et al 1997 IOVS). In a more recent study, a hypoxia response promoter was shown to upregulate the expression of a reporter gene in the region of CNV in an animal model. In addition, there is data to suggest that choroidal blood flow may be altered in patients with AMD (Grunwald et al 1998; Ross et al 1998), and this would lead to a hypoxic stimulus. Another possible source of hypoxia is diffuse thickening of Bruch's membrane with lipophilic material, thus decreasing diffusion of oxygen from the choroids to the RPE and the retina.

It is important to target the appropriate cell population with a gene therapy strategy. In the case of AMD and PDR this would ideally be cells of the retinal pigment epithelium (RPE) or those of the outer retina such as the photoreceptor (PR) cell layer that are specifically affected by the neovascularisation.

Hypoxia is a powerful regulator of gene expression in a wide range of different cell types (Wang and Semenza, 1993a). Studies of the genes involved in vascularization and carbohydrate metabolism have identified a number of genes that are induced under hypoxic conditions. For example, the gene encoding erythropoietin is known to be regulated by hypoxia.

Erythropoietin (EPO) is a hormone that regulates erythropoiesis, and hence, blood oxygen content. Cis-activating DNA sequences that function as tissue-specific hypoxia-inducible enhancers of human erythropoietin expression have been identified (Semenza et al., 1991). These enhancers contain regulatory elements, termed hypoxia response elements (HREs), which are the binding sites for hypoxia-inducible transcription factors such as hypoxia inducible factor-1 (HIF-1). A DNA enhancer sequence located 3′ to the mouse erythropoietin gene has been shown to confer oxygen-regulated expression on a variety of heterologous promoters (Pugh et al., 1991).

A second example of a hypoxia-associated regulatory sequence is an enhancer element that lies 5′ to the mouse phosphoglycerate kinase (PGK) gene promoter. The sequence of the enhancer has been published (McBurney et al., 1991) and its hypoxia-inducible properties disclosed in WO-A-95/21927. The HRE within this PGK enhancer has been defined as an 18 bp sequence.

Dachs et al. (1997) used a multimeric form of the mouse PGK-1 gene (Firth et al., 1994) to control expression of both marker and therapeutic genes in response to hypoxia in vitro and within solid tumours in vivo (Dachs et al, 1997).

Thus, since ischaemia is a general feature of solid tumours regardless of their cellular origin or patient population, it is possible to exploit tumour hypoxia to obtain selective expression of genes in tumours (Dachs et al., 1997).

Ischaemic damage may also occur in many other tissues when the blood supply to the tissue is reduced or cut off. Stroke, deep vein thrombosis, pulmonary embolus and renal failure are examples of conditions that can cause such damage. The cell death of cardiac tissue, called myocardial infarction, is due in large part to tissue damage caused by ischemia and/or ischemia followed by reperfusion. Recurrent ischaemia and reperfusion typically results in oxidative damage to cells from reactive oxygen species. The extent and type of damage depends on the severity and nature of the hypoxic stress. For example, the stress may cause tissue necrosis. Alternatively, the stress may initiate apoptosis (programmed cell death) to eliminate the damaged cells.

Ideally, a hypoxia-inducible expression construct for use in gene therapy should be capable of directing high level expression under hypoxic conditions whilst only directing low basal levels of transcription under normoxic conditions. This combination allows maximum delivery of therapeutic product to target cells without affecting normal surrounding oxygenated tissue.

In diseases involving hypoxic and ischaemic conditions such as PDR and AMD, alternative treatment paradigms are needed to replace current treatments having limited application and requiring repeat administration. Both argon laser and photodynamic therapy are inherently destructive and only delay vision loss. Therefore, there is an unmet need for therapies of ocular neovascularization that are less invasive, longer lasting, and/or physiologically controlled.

### SUMMARY OF THE INVENTION

The present invention provides a method for treating ocular neovascularization comprising delivering to target cells in the eye of a subject in need of treatment, a vector comprising a promoter sequence in operable linkage with a polynucleotide sequence encoding an angiostatic gene product, wherein the angiostatic gene product is expressed in the target cells, thereby treating ocular neovascularization in the subject.

The target cells are preferably retinal cells, more preferably retinal pigment epithelial cells, and the vector is preferably delivered to the target cells via direct sub-retinal injection

The promoter sequence can be a physiologically regulated promoter sequence, such as a hypoxically responsive promoter sequence, preferably a hypoxic response element (HRE). Details of HREs can be found in U.S. Pat. Nos. 5,942,434; and 6,265,390. Alternatively, the promoter sequence can be a constitutive promoter sequence, such as a CMV promoter, or a tissue specific promoter.

The ocular neovascularization can be choroidal or retinal, and can result in proliferative diabetic retinopathy (PDR) or age-related macular degeneration (AMD) in the subject.

The invention also provides a vector comprising a hypoxically regulated promoter sequence in operable linkage with a polynucleotide sequence encoding an angiostatic gene product.

The vector can be a viral vector, preferably a retroviral vector or an adeno-associated viral vector. The retroviral vector can be a lentiviral vector, preferably an EIAV-based lentiviral vector. Details of EIAV-based lentiviral vectors can be found in U.S. Pat. Nos. 6,277,633 and 6,521,457. Details of a minimal lentiviral vector sytem can be found in U.S. Pat. Nos. 6,669,936, 6,312,682, and 6,312,683.

The vector can further comprise a polynucleotide sequence encoding at least one additional angiostatic gene product.

The angiostatic gene product and the additional angiostatic gene product can be selected from the group consisting of endostatin, angiostatin, vascular endothelial growth factor receptor 1 (VEGFR1), FLT-1, and pigment epithelium-derived factor (PEDF). In a preferred embodiment, the angiostatic gene product is endostatin, and the additional angiostatic gene product is angiostatin.

In addition, the present invention provides lentiviral vectors that are responsive to hypoxia and to agents that mimic hypoxia. This regulation can be harnessed in vitro to enhance production of the vector and it can be used in vivo to regulate gene expression in response to a physiological signal. Such vectors have utility in a wide range of diseases where ischaemia, including hypoxia, is a feature, for example, cardiovascular disease, peripheral arterial disease, cancer, arthritis, and ocular diseases such as PDR and AMD.

The present invention further provides for polynucleotide constructs or vectors comprising at least two HRE repeats, and preferably at least three HRE repeats. Advantageously, the HRE repeats may be operably linked to a viral promoter. Preferably the HRE is a phosphoglycerate kinase (PGK) HRE. In a preferred embodiment the polynucleotide of the invention comprises three repeats of a PGK. Preferably the HRE repeats are operably linked to an SV40 promoter, a Moloney murine leukaemia virus (MoMLV) promoter, or a CMV promoter. Preferably, the HRE repeats are direct repeats. More preferably, the repeats are in their naturally occurring orientation.

In another embodiment, the present invention provides a polynucleotide construct or vector comprising at least three repeats of a phosphoglycerate kinase (PGK) hypoxia response element (HRE), operably linked to an SV40 promoter, a Moloney murine leukaemia virus (MoMLV) promoter, or a CMV promoter. Preferably, the HRE repeats are direct repeats. More preferably, the repeats are in their naturally occurring orientation.

A preferred functional HRE of the present invention consists of a nucleotide sequence as shown in SEQ ID NO:1 or SEQ ID NO:2. A preferred HRE/promoter construct of the invention comprises a nucleotide sequence as shown in SEQ ID NO:3, SEQ ID NO:4, or SEQ ID NO:5.

The present invention further provides for a polynucleotide construct or vector of the invention, operably linked to a nucleic acid of interest. (NOI), to produce in effect an expression cassette, such that the polynucleotide directs expression of the NOI in a host cell. Thus, the present invention further provides a polynucleotide construct or vector of the invention, operably linked to an NOI, for use in expressing the NOI in a host cell, for example a mammalian cell, more preferably a human cell. The NOI may also preferably be selected from polynucleotide sequences encoding proteins involved in the regulation of cell division, enzymes involved in cellular metabolic pathways, transcription factors and heat shock proteins. More preferably, the NOI may be an antisense sequence, an siRNA sequence, or a polynucleotide sequence encoding a growth factor, a receptor, an antibody, a cytokine, an apoptotic/cytotoxic enzyme/protein, a selectable marker, or an enzyme. Most preferably, the NOI is a polynucleotide sequence encoding an angiostatic gene, such as endostatin and/or angiostatin. The nucleotide sequences of both endostatin and angiostatin are known in the art. For example, the sequence of endostatin can be found in U.S. Pat. No. 6,544,758, and in U.S. Publication No. 20030087393. The sequence of angiostatin can be found in U.S. Pat. Nos. 5,792,845, 5,885,795 and 6,521,439.

The present invention also provides an autoregulatory cassette, wherein a further NOI is a polynucleotide sequence encoding an HIF-1α polypeptide that improves the induction under hypoxic conditions.

In a preferred embodiment, the present invention provides polynucleotide constructs or vectors described above that are viral vectors. Preferably the viral vector is a retroviral, such as a lentiviral, an adenoviral or a herpes simplex viral vector. More preferably the viral vector is a lentiviral vector. Most preferably, the lentiviral vector is an equine infectious anemia virus (EIAV) vector.

Polynucleotide constructs or vectors of the present invention may advantageously be used to deliver therapeutic products to mammalian cells. In particular, mammalian cells under hypoxic conditions will preferentially express the NOI under the control of the HRE/promoter construct, whereas surrounding cells under conditions of normoxia will express the NOI at much lower levels. In a preferred embodiment, the NOI encodes an angiostatic gene product. In a more preferred embodiment the angiostatic gene product is endostatin and/or angiostatin.

Accordingly, the present invention provides the use of the polynucleotide constructs or vectors described above for use in a method of treatment of a human or animal patient suffering from a disease in which hypoxia is a cause or a symptom or is otherwise present. In a preferred embodiment, the disease in which hypoxia is a cause or symptom or is otherwise present is a disease associated with ocular neovascularization such as PDR or AMD.

The present invention also provides a pharmaceutical composition comprising polynucleotide constructs of vectors described above together with a pharmaceutically acceptable carrier or diluent.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 shows a schematic diagram of the minimal EIAV-OBHRE vector based on the pONY8.4 EIAV vector series.

FIGS. 2A and 2B show ARPE-19 cells transduced with the EIAV-OBHRE recombinant LentiVector, under normoxic (2A) and hypoxic (2B) conditions.

FIGS. 3A and 3B show trans-retinal sections before (3A) and after (3B) laser induced rupture of Bruch's membrane to show the relative layers of the retina and choroid in relation to the neovascularisation. The new vessels extending into the subretinal space from the choroid are shown in green by immunostaining with an anti vWF antibody. FIGS. 3C and 3D show GFP reporter gene expression in the RPE (3C) using the pONY8.0 series of EIAV vectors pseudotyped with the VSV G envelope in the RPE and PR layers (3D) using the pONY8.4 series of EIAV vectors following subretinal delivery the EIAV LentiVector carrying the GFP reporter gene downstream of a CMV promoter.

FIGS. 4A and 4B show transretinal sections of murine retina following subretinal delivery of an EIAV-OBHRE LacZ virus and laser injury. LacZ expression is highlighted using green fluorescent antibody staining, which is only detected in isolated regions of the RPE and PR cell layers.

FIG. 5 shows codon optimization of human endostatin cDNA. The top line is the nucleotide sequence of codon optimized endostatin (SEQ ID NO:6). The middle line is the nucleotide sequence of native endostatin (SEQ ID NO:7). The bottom line is the amino acid sequence of endostatin (SEQ ID NO:8). The underlined region represents the tissue type plasminogen activator (TPA) signal sequence.

FIG. 6 shows codon optimized angiostatin. The top line is the nucleotide sequence of codon optimized angiostatin (SEQ ID NO:9). The middle line is the nucleotide sequence of native angiostatin (SEQ ID NO:10). The bottom line is the amino acid sequence of angiostatin (SEQ ID NO:11). The underlined region represents the tissue type plasminogen activator (TPA) signal sequence.

FIG. 7 shows a schematic diagram of the pONYK9.1mcs EIAV genome plasmid and the endostatin IRES angiostatin genome plasmid.

FIG. 8 shows a diagram of examples of the Retinostat EIAV genome series. The pRS1 genome is the constitutive (CMV) and the pRS1HRE is the hypoxia regulated (OBHRE) EIAV genome. The IRES is from EMCV.

FIG. 9 shows a schematic diagram of EIAV lentiviral vector production.

### DETAILED DESCRIPTION OF THE INVENTION

A. Polynucleotides

Polynucleotides of the invention may comprise DNA or RNA. They may be single-stranded or double-stranded. They may also be polynucleotides that include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out, for example, to enhance the in vivo activity or life span of polynucleotides of the invention.

1. Promoter/Enhancer Sequences

In one embodiment, the polynucleotide of the invention can comprise a hypoxia response element (HRE) operably linked to a promoter. An HRE for use in the present invention is a minimal nucleotide sequence that acts under conditions of hypoxia on a promoter sequence to which it is operably linked to increase the rate of transcription of a nucleotide sequence of interest (NOI) under the control of the promoter. Preferably, the HRE and promoter used in the present invention are selected such that expression of an NOI operably linked to these elements is minimal in the presence of a healthy supply of oxygen, and is upregulated under hypoxic or anoxic conditions. More preferably, the ratio of the rate of transcription under normoxic conditions (for example 21% oxygen) to the rate of transcription under hypoxic conditions (for example less than 1% oxygen or 0.1% oxygen) is at least 20:1, more preferably at least 50:1 or 100:1.

The polynucleotide of the present invention may comprise one or more HREs, preferably at least two or three. Where more than one HRE is used, they are typically spaced apart by at least about 4 to 6, more preferably at least about 6 nucleotides. In an especially preferred embodiment, at least two of the HREs are spaced apart by at least about 20 nucleotides, more preferably at least about 30 or 35 nucleotides. Spacing is typically measured from the 3′ end of the hypoxia inducible factor (for example HIF-1) binding site within one HRE to the 5′ end of the hypoxia inducible factor binding site of the next HRE. The HIF-1 binding site has the consensus C/A.G.G/C.ACGT.G/C/A (or its complement). In one embodiment, the spacer comprises the sequence 5′ GTCGTGCAGGCA 3′ (SEQ ID NO:12), or its complement, more preferably the sequence 5′TCTAGTGTCGTGCAGGCATCTAGT 3′ (SEQ ID NO:13), or its complement.

Multiple HRE repeats may be in the same or different orientation. In a preferred embodiment the HREs are in the same orientation, in particular, in the same orientation as found in the naturally occurring regulatory control sequence from which they originate, where appropriate. HREs may be upstream of the promoter (5′) or downstream of the promoter (3′) or both.

A variety of HREs are known in the art. For example, HREs have been found in association with a number of genes, including the erythropoietin (EPO) gene (Madan et al., 1993; Semenza and Wang, 1992). Other HREs have been isolated from regulatory regions of the muscle glycolytic enzyme pyruvate kinase (PKM) gene (Takenaka et al., 1989), the human muscle-specific β-enolase (ENO3) gene (Peshavaria and Day, 1991) and the endothelin-1 (ET-1) gene (Inoue et al., 1989). A list of HREs is given in Table 1 below. A particularly preferred HRE is the phosphoglycerate kinase (PGK) HRE (WO95/21927), such as the mouse PGK HRE P24 sequence CGCGTCGGTGCAGGACGTGACAAAT (SEQ ID NO:14) or its truncated P18 version GTCGGTGCAGGACGTGACA (SEQ ID NO:15). Another particularly preferred HRE is the D2 HRE—GTCGTGCAGTACGTGACA (SEQ ID NO:16).

A particular example of a hypoxia regulated enhancer is a binding element for transcription factor HIF-1 (Dachs et al., 1997; Wang and Semenza, 1993a; Firth et al., 1994) and/or a binding element for endothelial PAS domain protein (EPAS). In a particularly preferred embodiment, an HRE for use in the present invention is capable of binding both HIF-1 and E-PAS. However, an HRE for use in the present invention may be capable of preferentially binding E-PAS as opposed to HIF-1. Thus, an HRE for use in the present invention may bind E-PAS at least about 10 times, preferably at least about 20, 30, 40 or 50 times more strongly than HIF-1. This can be tested by, for example, in vitro binding assays such as electrophoretic band shift assays or by competition assays using nucleotides comprising HRE sites immobilised on a solid substrate such as sepharose beads.

TABLE 1
HREs

| | | |
|---|---|---|
| hEPO | GGGCCCTACGTGCTGTCTCACACAGC | (SEQ ID NO:17) |
| mEPO | GGGCCCTACGTGCTGCCTCGCATGGC | (SEQ ID NO:18) |
| mPGK | CGCGTCGTGCAGGACGTGACAAAT | (SEQ ID NO:19) |
| mLDH | CCAGCGGACGTGCGGGAACCCACGTGTAGG | (SEQ ID NO:20) |
| Glucose trpt | TCCACAGGCGTGCCGTCTGACACGCA | (SEQ ID NO:21) |
| hVEGF | CCACAGTGCATACGTGGGCTCCAACAGGTCCTCTT | (SEQ ID NO:22) |
| rVEGF | ACAGTGCATACGTGGGCTTCCACA | (SEQ ID NO:23) |
| hNOS | ACTACGTGCTGCCTAGG | (SEQ ID NO:24) |
| hAldolase | CCCCTCGGACGTGACTCGGACCACAT | (SEQ ID NO:25) |
| hEnolase | ACGCTGAGTGCGTGCGGGACTCGGAGTACGTGACGGA | (SEQ ID NO:26) |
| mHeme Oxygenase | CGGACGTGCTGGCGTGGCACGTCCTCTC | (SEQ ID NO:27) |

Where an EPAS-binding HRE is used, it may be desirable to enhance hypoxia specificity/inducibility by introducing into the host cell an NOI encoding the EPAS. Alternatively, the host cell used may naturally express the EPAS (for example epithelial cells, including tumour cells, and macrophage cells).

By contrast, where a HIF-1 binding HRE is used, it may be desirable to enhance hypoxia specificity/inducibility by introducing into the host cell an NOI encoding HIF-1, such as HIF-1α, under the control of the HRE by way of an autoregulatory system.

It is also possible to modify a known HRE, or design a novel HRE based on the HRE consensus sequence, to obtain an HRE suitable for use in the present invention provided that it is able to confer hypoxia-inducible transcription, typically when combined with a promoter sequence, or an operably linked NOI. Modifications include nucleotide substitutions, deletions and insertions.

An HRE may also contain additional sequences, for example, those with which it is naturally associated as part of an enhancer, or other sequences.

The level of expression of one or more NOIs under the control of a particular polynucleotide of the invention may be modulated by manipulating the enhancer/promoter region. For example, different domains within a promoter region may possess different gene regulatory activities. The roles of these different regions are typically assessed using vector constructs having different variants of the promoter with specific regions deleted (that is, deletion analysis). This approach may be used to identify, for example, the smallest region capable of conferring tissue specificity or the smallest region conferring hypoxia inducibility.

Promoters used in the polynucleotides of the invention are preferably strong promoters such as viral promoters. For example, strong viral promoters include the SV40 promoter, the cytomegalovirus (CMV) promoter, the rous sarcoma virus (RSV) promoter and murine leukaemia virus (MLV) promoters. Particularly preferred promoters are the SV40 and MoMLV promoters. Thus preferred HRE/promoter constructs include the constructs shown in SEQ ID NOs:3, 4 and 5.

However, in another preferred embodiment, particularly where a viral promoter and a HIF-1 autoregulated construct is used, it may be desirable to reduce basal transcription by using a promoter that lacks one or more of the transcriptional regulatory sequences normally associated with the TATA box or initiator sequence of the promoter. For example the promoter may lack a CAAT box motif, and/or an Sp1 consensus binding site, such as is normally found within the SV40 promoter. It may also be possible to use a minimal promoter consisting essentially of a TATA box or initiator linked to an HRE.

In addition to the HRE sequences and promoter, the polynucleotide of the invention may comprise additional regulatory control sequences. For example, additional levels of transcriptional control may be used to ensure that expression directed by the polynucleotide of the invention is confined to certain cell types or under certain conditions. Thus, additional enhancers may be operably linked to the polynucleotide of the invention, either downstream, upstream or both.

The additional regulatory sequence may be a sequence found in eukaryotic genes. For example, it may be a sequence derived from the genome of a cell in which expression of the NOI is to occur. Subject to the over-riding control of the HRE enhancer/promoter construct of the invention, the additional regulatory sequence may function to confer ubiquitous expression or alternatively tissue-specific expression. It is particularly preferred that additional regulatory sequences are used that are preferentially active in one or more specific cell types—such as any one or more of macrophages, endothelial cells or combinations thereof. Further examples include respiratory airway epithelial cells, hepatocytes, muscle cells, cardiac myocytes, synoviocytes, primary mammary epithelial cells and post-mitotically terminally differentiated non-replicating cells such as macrophages.

Examples of sequences that are cell specific include a macrophage-specific promoter or enhancer such as a CSF-1 promoter-enhancer, or elements from a mannose receptor gene promoter-enhancer. Alternatively, elements that are preferentially active in neutrophils, or a lymphocyte-specific enhancer such as an IL-2 gene enhancer, may be used. A particularly preferred tissue specific regulatory sequence is an HIV-derived sequence that confers macrophage-specific expression such as the XiaMac promoter (SEQ ID NO:28).

Another example is an endothelium specific promoter, which may be used to restrict the expression of an NOI to vascular endothelium. In particular, the correct choice of regulatory sequences can restrict expression to the neo-vasculature, that is, for example, specific to tumours. Jaggar et al. (1997) have described the use of the E-selectin and KDR promoters to express therapeutic genes from retroviral vectors specifically in endothelial cells. Hypoxia-regulated, endothelium specific promoters may be particularly useful for the delivery of anti-angiogenic factors to the vasculature endothelium.

The term “tissue specific” means a regulatory control sequence which is not necessarily restricted in activity to a single tissue type but which nevertheless shows selectivity in that it may be active in one group of tissues and less active or silent in another group.

Another example of a tissue specific promoter is the VMD2 promoter, which is capable of directing retinal pigment epithelium (RPE)-specific expression of an NOI (Epub ahead of print, Esumi et al. J Biol Chem (2004 Feb 24).

A number of tissue specific enhancers and promoters, for example as described above, may be particularly advantageous in practicing the present invention. In most instances, these enhancers may be isolated as convenient restriction digestion fragments suitable for cloning in a selected vector. Alternatively, enhancer or promoter fragments may be isolated using the polymerase chain reaction. Cloning of the amplified fragments may be facilitated by incorporating restriction sites at the 5′ end of the primers. Enhancer fragments may also be synthesized using, for example, solid-phase technology.

A particular example of a regulated sequence that may be added to the HRE/promoter of the invention to restrict expression to macrophages under defined conditions is the IRF system (Taniguchi et al., 1995; Kuhl et al., 1987). In this situation, an interferon response element (IRE) is used that can bind the transcription factors IRF1 and IRF2. IRF 2 is constitutively bound to this IRE in macrophages and lacks the ability to activate transcription. Interferon activates IRF1 expression that can then compete for binding with IRF2. The ability of IRF1 to activate transcription thereby reverses IRF2 repression. IRF1 also induces IRF2 gene expression thereby limiting activation of transcription by ‘auto shut off’. Inclusion of a tetramer of the IRE can block SV40 promoter function in the absence of IRF 1 activation.

Inclusion of this tetrameric sequence downstream of the HRE 5′ to the ATATAA (i.e. the TATA box like element in the SV40 promoter) confers repression in the absence of activation by interferon.

Interferon gamma is naturally present in inflammatory responses, including the response to tumours. Alternatively, interferon gamma can be provided exogenously, as a protein or as a gene, and delivered as a gene therapy. In a particular aspect of the invention, an autocrine regulatory circuit can provide interferon gamma. In this case, a simple HRE promoter, such as OBHRE, is linked to interferon gamma coding sequence. A second gene, for example a pro-drug activating enzyme or any gene from the above list, is linked to the XiaMac promoter that contains an IRF-1 responsive sequence (SEQ ID NO:29). The promoter is inactive in all cells, including macrophages. Upon exposure to hypoxia in the pathological condition, the interferon gamma is expressed. The expression of the therapeutic gene is then activated by the macrophage specific factors and the hypoxia responsive factors. This two-phase strategy can be applied to any repressor protein.

Additional regulatory sequences may also comprise elements that respond to specific stimuli, for example elements that bind steroid hormone receptors.

It may also be desirable to include regulatory elements that are inducible, for example, such that expression can be regulated by administration of exogenous substances. In this way, levels of expression of the NOI can be regulated during the life-time of the cell. “Inducible” means that the levels of expression obtained using the promoter can be regulated. For example, in a preferred embodiment, a polynucleotide of the invention comprises regulatory sequences responsive to the tet repressor/VP16 transcriptional activator fusion protein previously reported (Gossen and Bujard, 1992; Gossen et al, 1995). A second polynucleotide would then typically comprise a strong promoter (e.g. the CMV IE promoter) driving the expression of the tet repressor/VP16 fusion protein. Thus, in this example, expression of the first NOI would depend on the presence or absence of tetracycline.

A desirable characteristic of the polynucleotides of the present invention is that they possess a relatively low transcriptional activity in the absence of activated hypoxia-regulated enhancer elements, even in the target tissue. For example, “silencer” elements may be used which suppress the activity of a selected promoter in the absence of hypoxia. Another strategy is to use a HIF-1 autoregulated construct as described above and in the Examples.

Most of the above systems rely on transcription control; however it is quite possible to control protein expression at the translational level depending on the nature of the RNA transcript. In addition, it has been known for several years now that it is possible to select, from random pools, RNA sequences of 20 to 40 nucleotides, that bind quite tightly to a specific ligand used in the selection process. (See A D Ellington and J W Szostak “In vitro selection of RNA molecules that bind specific ligands” Nature 1990 346: 818-822; R R White, B A Sullenger and C R Rusconi “Developing aptamers into therapeutics” J. Clin Invest. 2000 106: 929-934.). Interestingly, the major applications seen for such observations has been the use of the RNA molecules as antagonists for various interactions occuring in cells, such as the HIV tat—TAR RNA interaction that facilitates HIV infection (White et al op.cit.). One publication has suggested using this mechanism as a way to control gene expression by inserting the aptamer into a message sequence then adding a cell permeable ligand for which the aptamer has been selected (G Werstuck and M R Green “Controlling Gene expression in living cells through small molecule-RNA interactions” Science (1998) 282:296-298 and WO00/20040). However, the molecules proposed for use were either aminoglycoside antibiotics such as kanamycin and tobramycin or Hoechst dyes. Thus the system does not propose to use innocuous compounds for this purpose but rather compounds with known toxicities or that have no history of human use. This system is therefore subject to issues described above. It also shows effects at concentration of drugs in the hundreds of micromolar to millimolar range. Typically this is the kind of concentration that is extremely difficult to reach in patient tissue or blood stream by oral administration of small molecule drugs.

It is possible to avoid these problems by selecting from a large library of sequences, with many more rounds of selection (20 to 40), aptamers that bind to innocuous well-characterized compounds with a record of human use. Ideally these are orally available, with known pharmacokinetics with a T½>12 h. These compounds are selected to be able to enter the tissue where it is desired to control expression. For example, for neural tissue the known ability to cross the blood brain barrier is important. The aptamer sequence is then inserted in the gene, the expression of which is to be controlled, and the safe permeable molecule used to turn off protein expression as desired. Examples of such small drug molecules include prescription drugs such as tetracycline or doxycycline, but also many over the counter (OTC) drugs (see such as aspirin or other mild analgesics), or compounds on the FDA list of “generally recognized as safe” (GRAS) compounds by the FDA. Other examples are nicotine (normally used to quit smoking) and other nucleoside analogues, and various food additives including color dyes etc. If single aptamer sequences are responsive but only partially suppress expression, multiple copies can be inserted. The gene, the expression of which is to be controlled, can in genral be delivered to animals and patients by any of the available viral or non-viral vector systems. (See “The development of Human Gene Therapy” T. Friedmann Ed., Cold Spring Harbor Laboratory Press, 1999.) It can be used to control expression of a therapeutic gene, an accessory gene such as a selectable marker or expression of a viral protein of a viral vector. In the case of a viral vector this can also be used to create replicating vectors, the replication of which is controllable by administration of an outside agent.

2. Nucleic Acids of Interest (NOI)

The polynucleotide of the invention is typically operably linked to an NOI, usually a heterologous gene. The term “heterologous gene” encompasses any gene. The heterologous gene may be any allelic variant of a wild-type gene, or it may be a mutant gene. The term “gene” is intended to cover nucleic acid sequences that are capable of being transcribed. Thus, sequences encoding mRNA, tRNA and rRNA are included within this definition. The sequences may be in the sense or antisense orientation with respect to the promoter. Antisense constructs can be used to inhibit the expression of a gene in a cell according to well-known techniques. Nucleic acids may be, for example, ribonucleic acid (RNA) or deoxyribonucleic acid (DNA) or analogues thereof. Sequences encoding mRNA will optionally include some or all of 5′ and/or 3′ transcribed but untranslated flanking sequences naturally, or otherwise, associated with the translated coding sequence. It may optionally further include the associated transcriptional control sequences normally associated with the transcribed sequences, for example transcriptional stop signals, polyadenylation sites and downstream enhancer elements. Nucleic acids may comprise cDNA or genomic DNA (which may contain introns). However, it is generally preferred to use cDNA because it is expressed more efficiently since intron removal is not required.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequence.

In accordance with the present invention, suitable NOI sequences include those that are of therapeutic and/or diagnostic application such as, but not limited to, sequences encoding angiostatic proteins, cytokines, chemokines, hormones, antibodies, engineered immunoglobulin-like molecules, single chain antibodies, fusion proteins, enzymes, immune co-stimulatory molecules, immunomodulatory molecules, anti-sense RNA, transdominant negative mutants of target proteins, toxins, conditional toxins, antigens, tumour suppressors growth factors, membrane proteins, vasoactive proteins and peptides, anti-viral proteins and ribozymes, and derivatives thereof (such as with an associated reporter group).

Suitable NOIs for use in the present invention in the treatment or prophylaxis of diseases having an ischaemic-associated pathology, such as diabetic retinopathies, age-related macular degeneration and cancers, include NOIs encoding proteins that destroy the target cell (for example a ribosomal toxin); or which act as tumour suppressors (such as wild-type p53), activators of anti-tumour immune mechanisms (such as cytokines, co-stimulatory molecules and immunoglobulins), or inhibitors of angiogenesis or angiostatic proteins; or which provide enhanced drug sensitivity (such as pro-drug activation enzymes); or which indirectly stimulate destruction of a target cell by natural effector cells, for example, a strong antigen to stimulate the immune system or convert a precursor substance to a toxic substance which destroys the target cell (for example a prodrug activating enzyme). Encoded proteins could also destroy bystander tumour cells (for example with secreted antitumour antibody-ribosomal toxin fusion protein), indirectly stimulated destruction of bystander tumour cells (for example cytokines to stimulate the immune system or procoagulant proteins causing local vascular occlusion) or convert a precursor substance to a toxic substance which destroys bystander tumour cells (e.g. an enzyme which activates a prodrug, to a diffusible drug).

NOI(s) may be used which encode antisense transcripts or ribozymes that interfere with expression of cellular genes for tumour persistence (for example against aberrant myc transcripts in Burkitts lymphoma or against bcr-abl transcripts in chronic myeloid leukemia.

The use of combinations of such NOIs is also envisaged.

Instead of, or as well as, being selectively expressed in target tissues, the one or more NOIs may encode a pro-drug activation enzyme or enzymes which have no significant effect or no deleterious effect until the individual is treated with one or more pro-drugs upon which the enzyme or enzymes act. In the presence of the active NOI, treatment of an individual with the appropriate pro-drug leads to enhanced reduction in tumour growth or survival.

A pro-drug activating enzyme may be delivered to a tumour site for the treatment of a cancer. In each case, a suitable pro-drug is used in the treatment of the patient in combination with the appropriate pro-drug activating enzyme. An appropriate pro-drug is administered in conjunction with the vector. Examples of pro-drugs include: etoposide phosphate (with alkaline phosphatase); 5-fluorocytosine (with cytosine deaminase); doxorubicin-N-p-hydroxyphenoxyacetamide (with penicillin-V-amidase); para-N-bis(2-chloroethyl) aminobenzoyl glutamate (with carboxypeptidase G2); cephalosporin nitrogen mustard carbamates (with β-lactamase); SR4233 (with P450 Reducase); ganciclovir (with HSV thymidine kinase); mustard pro-drugs with nitroreductase and cyclophosphamide (with P450).

Examples of suitable pro-drug activation enzymes for use in the invention include a thymidine phosphorylase, which activates the 5-fluoro-uracil pro-drugs capcetabine and furtulon; thymidine kinase from herpes simplex virus which activates ganciclovir; a cytochrome P450 which activates a pro-drug such as cyclophosphamide to a DNA damaging agent; and cytosine deaminase which activates 5-fluorocytosine. Preferably, an enzyme of human origin is used.

Suitable NOIs for use in the treatment or prevention of ischaemic heart disease include NOIs encoding plasminogen activators. Suitable NOIs for the treatment or prevention of rheumatoid arthritis or cerebral malaria include genes encoding anti-inflammatory proteins, antibodies directed against tumour necrosis factor (TNF) alpha, and anti-adhesion molecules (such as antibody molecules or receptors specific for adhesion molecules). Suitable NOIs for use in the treatment or prevention of diabetic retinopathies, AMD, or cancers include NOIs encoding angiostatic proteins, such as angiostatin and/or endostatin.

The expression products encoded by the NOIs may be proteins that are secreted from the cell. Alternatively, the NOI expression products are not secreted and are active within the cell. In either event, it is preferred for the NOI expression product to demonstrate a bystander effector or a distant bystander effect, wherein the production of the expression product in one cell leads to the killing of additional, related cells, either neighbouring or distant (e.g. metastatic), which possess a common phenotype.

Where macrophages or other haematopoietic cells (HSCs) are used, NOIs may be used which encode, for example, cytokines. These would serve to direct the subsequent differentiation of the HSCs containing a therapeutic NOI. Suitable cytokines and growth factors include but are not limited to: ApoE, Apo-SAA, BDNF, Cardiotrophin-1, EGF, ENA-78, Eotaxin, Eotaxin-2, Exodus-2, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1α, SCF, SCGF, stem cell factor (SCF), TARC, TGF-α, TGF-β, TGF-β2, TGF-β3, tumour necrosis factor (TNF), TNF-α, TNF-β, TNIL-1, TPO, VEGF, GCP-2, GRO/MGSA, GRO-β, GRO-γ and HCC1.

For some applications, a combination of some of these cytokines may be preferred, in particular, a combination that includes IL-3, IL-6 and SCF, for the maintenance and expansion of stem cell populations. For differentiation in vitro, further cytokines may be added such as GM-CSF and M-CSF to induce differentiation of macrophages or GM-CSF and G-CSF to obtain neutrophils. On reintroduction of the engineered cells into the individual from whom they were derived, the body's own mechanisms then permit the cells or their differentiated progeny to migrate into the affected area, e.g. the tumour.

Optionally, another NOI may be a suicide gene, the expression of which, in the presence of an exogenous substance, results in the destruction of the transfected or transduced cell. An example of a suicide gene includes the herpes simplex virus thymidine kinase gene (HSV tk), which can kill infected and bystander cells following treatment with ganciclovir.

Optionally another NOI may be a targeting protein (such as an antibody to the stem cell factor receptor (WO-A-92/17505; WO-A-92/21766). For example, recombinant (ecotropic) retroviruses displaying an antibody (or growth factor or peptide) against a receptor present on HSCs (CD34 or stem cell factor, for example) might be used for targeted cell delivery to these cells, either ex vivo by incubating unfractionated bone marrow with virus or by intravenous delivery of virus.

NOIs may also include marker genes (for example, encoding β-galactosidase or green fluorescent protein (GFP)) or genes whose products regulate the expression of other genes (for example, transcriptional regulatory factors, including the tet repressor/VP16 transcriptional activator fusion protein). In addition, NOIs may comprise sequences coding fusion protein partners in frame with a sequence encoding a protein of interest. Examples of fusion protein partners include the DNA binding or transcriptional activation domain of GAL4, a 6×His tag and β-galactosidase. It may also be desirable to add targeting sequences to target proteins encoding by NOIs to particular cell compartments or to secretory pathways. Such targeting sequences have been extensively characterized in the art.

In a preferred embodiment, at least one NOI, operably linked to an HRE/promoter of the present invention encodes a HIF-1 transcription factor or a homologue or fragment thereof capable of binding to a HIF-1 binding site. Such a construct will provide an autoregulated system since in the presence of hypoxia, expression of HIF-1 from the HRE construct will increase and serve to further increase transcription from the HRE construct and other HRE constructs present.

B. Enhancement of Hypoxic Response by Inhibiting VHL-Mediated Degradation of HIF

Maxwell et al., (1999) have published evidence that suggests the tumour suppressor protein VHL targets hypoxia-inducible factors for oxygen-dependent proteolysis. Prior to this, data has been published suggesting that in order to regulate hypoxia-inducible gene expression VHL requires binding to other proteins including elongins, B and C and Cu12 (Lonergan, 1998). Disruption of this multiprotein complex may be a route to block the degradation of HIFs and thereby increase longevity/degree of expression of HRE regulated genes. There are several possible methods for inhibiting VHL function.

A synthetic peptide derived from pVHL (for example amino acids 157-172) may be used to block the binding of full-length endogenous pVHL to Elongin B and C. Introduction of this peptide into cells by standard transfection techniques preferably as part of a lipid complex, may prevent association of endogenous VHL with the rest of the complex resulting in the failure to degrade HIF.

Alternatively, the VHL peptide may be co-expressed under the control of an HRE/promoter construct with another NOI. The optional use of an IRES sequence will allow both the VHL construct and NOI to be expressed under the control of one regulatory sequence.

Typically The VHL peptide, or other peptide that inhibits VHL function may be used in conjunction with an HRE autoregulated system where HRE controls expression of the therapeutic/reporter gene in concert with expression of a HIF transcription factor. The latter may also be coordinately regulated by use of an IRES sequence.

With respect to suitable peptides for inhibiting VHL function, the domain structure of proteins allows the design of proteins that retain certain features but are functionally silent for others, e.g. maintaining binding to one member of the complex but not to others. Expression of such a mutant protein would have a dominant negative effect on the normal protein-protein interaction i.e., would compete for binding with the wild type endogenous protein.

An example of this would be to express mutant VHL, which could be designed using natural mutations found in patients with VHL-associated disease e.g. renal cell ca, hemangioblastomas. This would result in a reduction in proteolytic targeting of HIF and, as a consequence, increased HRE-dependent gene expression. In nature, VHL mutation is frequently observed in the context of aberrant normoxic expression of HRE regulated genes such as those of the glycolytic enzymes.

Thus, in another preferred embodiment, the NOI encodes a polypeptide capable of inhibiting binding of the tumour suppressor protein VHL (Maxwell et al., 1999) to one or more of elongin B, elongin C and Cu12 (Lonergan, 1998). As discussed above, suitable polypeptides include variants of wild type VHL or its binding partners that can compete for binding to the multiprotein complex. Suitable polypeptides may be identified, for example, by in vitro binding assays using VHL and/or one of its binding partners. Other screening techniques include two hybrid screens to identify interacting sequences.

A particularly preferred peptide is a peptide consisting essentially of amino acids 157 to 172 of the full length VHL sequence.

Alternatively, the polypeptide capable of inhibiting binding of the tumour suppressor protein VHL to one or more of elongin B, elongin C and Cu12 may be introduced by other means, such as by separately transfecting a construct encoding the polypeptide into target cells, or by introducing pre-synthesised peptides.

Alternative approaches include expressing in a target cell inhibitory RNA molecules that effect the direct or indirect cleavage of VHL mRNA and/or precursor RNA. Inhibitory RNA molecules include antisense VHL constructs, ribozymes that cleave VHL mRNA and external guide sequences (EGSs). NOIs encoding the inhibitory RNA molecules may be introduced into nucleotide constructs, vectors and viral vectors of the present invention in a similar manner to that described above and below for other NOIs. Antisense VHL constructs are also included in a general sense since antisense inhibitory effects are mediated by mechanisms other than RNase cleavage. Thus, NOIs that encode inhibitory RNA molecules that bind to and prevent VHL RNA processing and/or expression, for example splicing, transport and/or translation, are also included.

Thus, in another embodiment of the invention, the NOI encodes (i) one or more inhibitory RNA molecules that effect the direct or indirect cleavage of VHL mRNA and/or precursor RNA or (ii) one or more inhibitory RNA molecules that bind to and prevent VHL RNA processing and/or expression.

C. Nucleic Acid Vectors

Polynucleotides of the invention can be incorporated into a recombinant replicable vector. The vector may be used to replicate the nucleic acid in a compatible host cell. Thus, in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli, yeast, mammalian cell lines and other eukaryotic cell lines, for example insect Sf9 cells.

A vector comprising a polynucleotide of the invention, which is operably linked to an NOI, can be considered to be an expression vector since, under suitable conditions, the NOI will be expressed under the control of the HRE/promoter construct of the present invention. However, it is not necessary for a vector of the invention to comprise an NOI. Nonetheless it is possible to introduce an NOI into the vector at a later stage. Thus a vector of the invention that lacks an NOI can be considered to be a cloning vector. Preferably, a cloning vector of the invention comprises a multiple cloning site downstream of the HRE/promoter sequences to enable an NOI to be cloned into the vector when required whereby it is then operably linked to the HRE/promoter sequences.

The vectors may be for example, plasmids, chromosomes, artificial chromosomes or virus vectors. The vectors may contain one or more selectable marker genes, for example an ampicillin resistance gene in the case of a bacterial plasmid or a neomycin resistance gene for a mammalian vector. Vectors may be used, for example, to transfect, transform or transduce a host cell either in vitro or in vivo.

Non-viral delivery systems include but are not limited to DNA transfection methods. Here, transfection includes a process using a non-viral vector to deliver a gene to a target mammalian cell. Typical transfection methods include electroporation, DNA biolistics, lipid-mediated transfection, compacted DNA-mediated transfection, liposomes, immunoliposomes, lipofectin, cationic agent-mediated, cationic facial anphiphiles (CFAs), multivalent cations such as spermine, cationic lipids or polylysine, 1,2,-bis (oleoyloxy)-3-(trimethylammonio) propane (DOTAP)-cholesterol complexes and combinations thereof.

Viral delivery systems include but are not limited to an adenovirus vector, an adeno-associated viral (AAV) vector, a herpes viral vector, a retroviral vector, such as a lentiviral vector, a baculoviral vector and combination vectors. In the case of viral vectors, gene delivery is typically mediated by viral infection of a target cell.

D. Viral Vectors

Retroviruses

The vector of the present invention can be a retroviral vector derived from or derivable from any suitable retrovirus. A large number of different retroviruses have been identified. Examples include: murine leukemia virus (MLV), human immunodeficiency virus (HIV), simian immunodeficiency virus, human T-cell leukemia virus (HTLV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). A detailed list of retroviruses may be found in Coffin et al., 1997, “Retroviruses”, Cold Spring Harbour Laboratory Press Eds: J M Coffin, S M Hughes, H E Varmus pp 758-763.

Details on the genomic structure of some retroviruses may be found in the art. By way of example, details on HIV and Mo-MLV may be found from the NCBI Genbank (Genome Accession Nos. AF033819 and AF033811, respectively).

Retroviruses may be broadly divided into two categories: namely, “simple” and “complex”. Retroviruses may even be further divided into seven groups. Five of these groups represent retroviruses with oncogenic potential. The remaining two groups are the lentiviruses and the spumaviruses. A review of these retroviruses is presented in Coffin et al., 1997 (ibid).

Each retroviral genome comprises genes called gag, pol and env which code for virion proteins and enzymes. In the provirus, these genes are flanked at both ends by regions called long terminal repeats (LTRs). The LTRs are responsible for proviral integration, and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

The basic molecular organization of an infectious retroviral RNA genome is (5′) R—U5-gag, pol, env—U3-R (3′). In a defective retroviral vector genome gag, pol and env may be absent or not functional. The R regions at both ends of the RNA are repeated sequences. U5 and U3 represent unique sequences at the 5′ and 3′ ends of the RNA genome respectively.

Host range and tissue tropism varies between different retroviruses. In some cases, this specificity may restrict the transduction potential of a recombinant retroviral vector. For this reason, many gene therapy experiments have used MLV. A particular MLV that has an envelope protein called 4070A is known as an amphotropic virus, and this can also infect human cells because its envelope protein “docks” with a phosphate transport protein that is conserved between man and mouse. This transporter is ubiquitous and so these viruses are capable of infecting many cell types.

In some cases however, it may be beneficial, especially from a safety point of view, to target specifically restricted cells. Replacement of the env gene with a heterologous env gene is an example of a technique or strategy used to target specifically certain cell types. This technique is called pseudotyping and examples may be found in WO-A-98/05759, WO-A-98/05754, WO-A-97/17457, WO-A-96/09400 and WO-A-91/00047.

The term “recombinant retroviral vector” (RRV) refers to a vector with sufficient retroviral genetic information to allow packaging of an RNA genome, in the presence of packaging components, into a viral particle capable of infecting a target cell. Infection of the target cell includes reverse transcription and integration into the target cell genome. The RRV in use typically carries non-viral coding sequences which are to be delivered by the vector to the target cell. An RRV is incapable of independent replication to produce infectious retroviral particles within the final target cell.

In a typical recombinant retroviral vector for use in gene delivery or therapy, at least part of one or more of the gag, pol and env protein coding regions essential for replication may be removed from the virus. This makes the retroviral vector replication-defective. The removed portions may even be replaced by an NOI to generate a virus capable of integrating its genome into a host genome but wherein the modified viral genome is unable to propagate itself due to a lack of structural proteins. When integrated in the host genome, expression of the NOI occurs—resulting in, for example, a therapeutic and/or a diagnostic effect. Thus, the transfer of an NOI into a site of interest is typically achieved by: integrating the NOI into the recombinant viral vector; packaging the modified viral vector into a virion coat; and allowing transduction of a site of interest—such as a targeted cell or a targeted cell population.

Replication-defective retroviral vectors are typically propagated, for example to prepare suitable titres of the retroviral vector for subsequent transduction, by using a combination of a packaging or helper cell line and the recombinant vector. That is to say, that the three packaging proteins can be provided in trans.

A “packaging cell line” contains one or more of the retroviral gag, pol and env genes. The packaging cell line produces the proteins required for packaging retroviral DNA but it cannot bring about encapsidation due to the lack of a psi region. However, when a recombinant vector carrying an NOI and a psi region is introduced into the packaging cell line, the helper proteins can package the psi-positive recombinant vector to produce the recombinant virus stock. This virus stock can be used to transduce cells to introduce the NOI into the genome of the target cells. It is preferred to use a psi packaging signal, called psi plus, that contains additional sequences spanning from upstream of the splice donor to downstream of the gag start codon (Bender et al., 1987) since this has been shown to increase viral titres.

The recombinant virus whose genome lacks all genes required to make viral proteins can tranduce only once and cannot propagate. These viral vectors, which are only capable of a single round of transduction of target cells, are known as replication defective vectors. Hence, the NOI is introduced into the host/target cell genome without the generation of potentially harmful retrovirus. A summary of the available packaging lines is presented in Coffin et al., 1997 (ibid).

Retroviral packaging cell lines in which the gag, pol and env viral coding regions are carried on separate expression plasmids that are independently transfected into a packaging cell line are preferably used. This strategy, sometimes referred to as the three-plasmid transfection method (Soneoka et al., 1995), reduces the potential for production of a replication-competent virus since three recombinant events are required for wild type viral production. As recombination is greatly facilitated by homology, reducing or eliminating homology between the genomes of the vector and the helper can also be used to reduce the problem of replication-competent helper virus production.

An alternative to stably transfected packaging cell lines is to use transient transfected cell lines. Transient transfections may advantageously be used to measure levels of vector production when vectors are being developed. In this regard, transient transfection avoids the longer time required to generate stable vector-producing cell lines and may also be used if the vector or retroviral packaging components are toxic to cells. Components typically used to generate retroviral vectors include a plasmid encoding the gag/pol proteins, a plasmid encoding the env protein and a plasmid containing an NOI. Vector production involves transient transfection of one or more of these components into cells containing the other required components. If the vector encodes toxic genes or genes that interfere with the replication of the host cell, such as inhibitors of the cell cycle or genes that induce apotosis, it may be difficult to generate stable vector-producing cell lines, but transient transfection can be used to produce the vector before the cells die. Also, cell lines have been developed using transient transfection that produce vector titre levels that are comparable to the levels obtained from stable vector-producing cell lines (Pear et al., 1993).

It is highly desirable to use high-titre virus preparations in both experimental and practical applications. Techniques for increasing viral titre include using a psi plus packaging signal as discussed above and concentration of viral stocks. In addition, the use of different envelope proteins, such as the G protein from vesicular-stomatitis virus has improved titres following concentration to 10⁹ per ml (Cosset et al., 1995).

In addition to manipulating the retroviral vector with a view to increasing vector titre, retroviral vectors have also been designed to induce the production of a specific NOI in transduced cells. As already mentioned, the most common retroviral vector design involves the replacement of retroviral sequences with one or more NOIs to create replication-defective vectors. With regard to regulation of expression of the NOI, there are three main approaches currently in use.

1. The simplest approach has been to use the promoter in the retroviral 5′ LTR to control the expression of a cDNA encoding an NOI or to alter the enhancer/promoter of the LTR to provide tissue-specific expression or inducibility. Where multiple NOIs are inserted, the additional downsteam NOIs can be expressed from a polycistronic mRNA by the use of internal ribosome entry sites.

2. The NOI may be operably linked to an internal heterologous promoter. This arrangement permits more flexibility in promoter selection. Additional NOIs can still be expressed from the 5′LTR or the LTR can be mutated to prevent expression following infection of a target cell.

3. The NOI is inserted together with regulatory control elements in the reverse orientation to the 5′LTR. Genomic sequences including enhancers, promoters, introns and 3′ regions may be included. In this way it may be possible to achieve tightly regulated tissue-specific gene expression.

In addition, we have now shown that there is a particular advantage to configuring retroviral vectors, in particular lentiviral vectors, as single transcription unit vectors whereby the HRE/promoter construct of the invention is placed within the 3′LTR such that the resultant duplication of the 3′LTR also leads to duplication of the regulatory sequence (i.e. the 5′LTR of the provirus will contain the HRE/promoter construct duplicated from the 3′LTR). We have now shown that this arrangement enhances the activated response to hypoxia in a synergistic manner. Consequently, it is preferred to use a retroviral vector that comprises an HRE/promoter of the invention within its LTR. More specifically, the HRE/promoter construct (optionally together with any additional regulatory sequences such as tissue-specific enhancer elements) may be inserted into the 3′ U3 region of the retroviral vector or the 5′ U5 region, most preferably the 3′ U3 region. Preferably, the NOI is not also inserted into the LTR since the resulting two copies in the provirus can decrease the size of the NOI, which can be accommodated by the retroviral vector. Instead, the NOI is preferably inserted into the region of the retroviral vector that is normally occupied by the env gene.

The NOI may or may not include a selectable marker. If the vector contains an NOI that is not a selectable marker, the vector can be introduced into packaging cells by co-transfection with a selectable marker present on a separate plasmid. This strategy has an appealing advantage for gene therapy in that a single protein is expressed in the ultimate target cells and possible toxicity or antigenicity of a selectable marker is avoided.

The current methodologies used to design retroviral vectors that express two or more proteins have relied on three general strategies. These include: (i) the expression of different proteins from alternatively spliced mRNAs transcribed from one promoter; (ii) the use of the promoter in the 5′ LTR and internal promoters to drive transcription of different cDNAs and (iii) the use of internal ribosomal entry site (IRES) elements to allow translation of multiple coding regions from either a single mRNA or from fusion proteins that can then be expressed from an open reading frame.

Vectors containing internal heterologous promoters have been widely used to express multiple genes. An internal promoter makes it possible to exploit promoter/enhancer combinations other than the viral LTR for driving gene expression. Multiple internal promoters can be included in a retroviral vector and it has proved possible to express at least three different cDNAs each from its own promoter.

The retroviral vector of the present invention can be a self-inactivating (SIN) vector system. By way of example, self-inactivating retroviral vector systems have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus. However, any promoter(s) internal to the LTRs in such vectors will still be transcriptionally active. This strategy has been employed to eliminate effects of the enhancers and promoters in the viral LTRs on transcription from internally placed genes. Such effects include increased transcription or suppression of transcription. This strategy can also be used to eliminate downstream transcription from the 3′ LTR into genomic DNA. This is of particular concern in human gene therapy where it may be important to prevent the adventitious activation of an endogenous oncogene. Yu et al., (1986) PNAS 83: 3194-98; Marty et al., (1990) Biochimie 72: 885-7; Naviaux et al., (1996) J. Virol. 70: 5701-5; Iwakuma et al., (1999) Virol. 261: 120-32; Deglon et al., (2000) Human Gene Therapy 11: 179-90.

Lentiviruses

The lentivirus group can be divided into “primate” and “non-primate”. Examples of primate lentiviruses include human immunodeficiency virus (HIV), the causative agent of human auto-immunodeficiency syndrome (AIDS), and simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

A distinction between the lentivirus family and other types of retroviruses is that lentiviruses have the capability to infect both dividing and non-dividing cells. In contrast, other retroviruses—such as MLV—are unable to infect non-dividing cells such as those that make up, for example, muscle, brain, lung and liver tissue. Thus, lentiviral vectors may advantageously be used in the present invention since lentiviruses are capable of infecting a wide range of non-dividing cells, by contrast to certain other retroviruses that require cell division for stable integration.

A number of vectors have been developed based on various members of the lentivirus sub-family of the retroviridae and a number of these are the subject of patent applications (WO-A-98/18815; WO-A-97/12622). Preferred lentiviral vectors are based on HIV, SIV or EIAV. The simplest vectors constructed from HIV-1 have the complete HIV genome except for a deletion of part of the env coding region or replacement of the nef coding region. Notably these vectors express gag/pol and all of the accessory genes hence require only an envelope to produce infectious virus particles. Of the accessory genes vif, vpr, vpu and nef are non-essential. Vectors have also been described that are efficient yet lack most or all of the accessory factors, for example Kim et al., 1998. Thus a lentiviral vector of the invention preferably lacks at least one accessory gene, more preferably all accessory genes.

It has been demonstrated that a lentivirus minimal system can be constructed from HIV, SIV, FIV, and EIAV viruses. Such a system requires none of the additional genes vif, vpr, vpx, vpu, tat, rev and nef for either vector production or for transduction of dividing and non-dividing cells. It has also been demonstrated that an EIAV minimal vector system can be constructed which does not require S2 for either vector production or for transduction of dividing and non-dividing cells. The deletion of additional genes is highly advantageous. Firstly, it permits vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO-A-99/32646 and in WO-A-98/17815.

Thus, preferably, the delivery system used in the invention is devoid of at least tat and S2 (if it is an EIAV vector system), and possibly also vif, vpr, vpx, vpu and nef. More preferably, the systems of the present invention are also devoid of rev. Rev was previously thought to be essential in some retroviral genomes for efficient virus production. For example, in the case of HIV, it was thought that rev and RRE sequence should be included. However, it has been found that the requirement for rev and RRE can be reduced or eliminated by codon optimisation or by replacement with other functional equivalent systems such as the MPMV system or the woodchuck post-transcriptional regulatory element (WPRE). As expression of the codon optimised gag-pol is REV independent, RRE can be removed from the gag-pol expression cassette, thus removing any potential for recombination with any RRE contained on the vector genome.

In a preferred embodiment the viral genome of the first aspect of the invention lacks the Rev response element (RRE).

In a preferred embodiment, the system used in the present invention is based on a so-called “minimal” system in which some or all of the additional genes have been removed.

In a preferred embodiment, the delivery system includes the WPRE for enhanced transgene expression. J. Virology 73: 2866-92, 1999.

Codon Optimisation

Codon optimisation has previously been described in WO99/41397. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including HIV and other lentiviruses, use a large number of rare codons, and by changing these to correspond to commonly used mammalian codons, increased expression of the packaging components in mammalian producer cells can be achieved. Codon usage tables are known in the art for mammalian cells, as well as for a variety of other organisms.

Codon optimisation has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. Codon optimisation also overcomes the Rev/RRE requirement for export, rendering optimised sequences Rev independent. Codon optimisation also reduces homologous recombination between different constructs within the vector system (for example between the regions of overlap in the gag-pol and env open reading frames). The overall effect of codon optimisation is therefore a notable increase in viral titre and improved safety.

In one embodiment only codons relating to INS are codon optimised. However, in a much more preferred and practical embodiment, the sequences are codon optimised in their entirety, with the exception of the sequence encompassing the frameshift site.

The gag-pol gene comprises two overlapping reading frames encoding gag and pol proteins respectively. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimised. Retaining this fragment will enable more efficient expression of the gag-pol proteins.

For EIAV the beginning of the overlap has been taken to be nt 1262 (where nucleotide 1 is the A of the gag ATG). The end of the overlap is at 1461 bp. In order to ensure that the frameshift site and the gag-pol overlap are preserved, the wild type sequence has been retained from nt 1156 to 1465.

Derivations from optimal codon usage may be made, for example, in order to accommodate convenient restriction sites, and conservative amino acid changes may be introduced into the gag-pol proteins.

In a highly preferred embodiment, codon optimisation was based on highly expressed mammalian genes. The third and sometimes the second and third base may be changed.

Due to the degenerate nature of the genetic code, it will be appreciated that numerous gag-pol sequences can be achieved by a skilled worker. Also there are many retroviral variants described which can be used as a starting point for generating a codon optimised gag-pol sequence. Lentiviral genomes can be quite variable. For example, there are many quasi-species of HIV-1 that are still functional. This is also the case for EIAV. These variants may be used to enhance particular parts of the transduction process.

The strategy for codon optimised gag-pol sequences can be used in relation to any retrovirus. This would apply to all lentiviruses, including EIAV, FIV, BIV, CAEV, VMR, SIV, HIV-1 and HIV-2. In addition this method could be used to increase expression of genes from HTLV-1, HTLV-2, HFV, HSRV and human endogenous retroviruses (HERV), MLV and other retroviruses.

Codon optimisation can render gag-pol expression Rev independent. In order to enable the use of anti-rev or RRE factors in the retroviral vector, however, it would be necessary to render the viral vector generation system totally Rev/RRE independent. Thus, the genome also needs to be modified. This is achieved by optimising vector genome components. Advantageously, these modifications also lead to the production of a safer system absent of all additional proteins both in the producer and in the transduced cell.

As described above, the packaging components for a retroviral vector include expression products of gag, pol and env genes. In addition, efficient packaging depends on a short sequence of 4 stem loops followed by a partial sequence from gag and env (the “packaging signal”). Thus, inclusion of a deleted gag sequence in the retroviral vector genome (in addition to the full gag sequence on the packaging construct) will optimise vector titre. To date, efficient packaging has been reported to require from 255 to 360 nucleotides of gag in vectors that still retain env sequences, or about 40 nucleotides of gag in a particular combination of splice donor mutation, gag and env deletions. It has surprisingly been found that a deletion of all but the N-terminal 360 or so nucleotides in gag leads to an increase in vector titre. Thus, preferably, the retroviral vector genome includes a gag sequence that comprises one or more deletions, more preferably the gag sequence comprises about 360 nucleotides derivable from the N-terminus.

E. Host Cells and Target Cells

Polynucleotide constructs, nucleic acid vectors and viral vectors of the invention may be introduced into a variety of host cells. Host cells include both prokaryotic, for example bacterial, and eukaryotic, for example yeast and higher eukaryotic cells (such as insect and mammalian, for example human, cells). Host cells may be used to propagate both non-viral and viral vectors, for example to prepare nucleic acid vectors comprising a polynucleotide of the invention or to prepare high titre viral stocks.

Alternatively, host cells comprising a polynucleotide of the invention/NOI may be used in therapy, such as the use of macrophages discussed below in, for example, ex vivo therapy.

Non-viral nucleic acids and viral vectors are typically introduced into host cells using techniques well known in the art such as transformation or transfection. Viral vectors may also be introduced into host cells using by infection.

Target cells, in the context of the present invention, means cells of, or from, an organism that typically it is desired to treat, rather than simply cell lines. Target cells may be removed from the organism and subsequently returned after treatment, or targeted in vivo. Thus for example, tumour cells in vivo can be considered to be target cells.

Examples of target cells include tumour cells, in particular, tumour cells under conditions of hypoxia. The target cell may be a growth-arrested cell capable of undergoing cell division such as a cell in a central portion of a solid tumour mass or a stem cell such as an HSC or a CD34⁺ cell. As a further alternative, the target cell may be a precursor of a differentiated cell such as a monocyte precursor, a CD33⁺ cell, or a myeloid precursor. The target cell may also be a differentiated cell such as a neuron, astrocyte, glial cell, microglial cell, macrophage, monocyte, epithelial cell, endothelial cell or hepatocyte. Target cells may be transfected or transduced either in vitro after isolation from an individual or may be transfected or transduced directly in vivo.

Preferred host cells/target cells include cells in which EPAS is expressed, more preferably cells in which EPAS is expressed but HIF-1 is not (or at much lower levels).

In a particularly preferred embodiment, haematopoietic stem cells such as macrophages are used as host cells/target cells. HSCs are pluripotent stem cells that give rise to all blood cell lineages in mammals. HSCs differentiate into various cell lineages under the influence of microenvironmental factors such as cell-to-cell interactions and the presence of soluble cell cytokines. Four major cell lineages arise from the HSCs. These include: erythroid (erythrocytes); megakaryocytic (platelets); myelbid (granulocytes and monocytes); and lymphoid (lymphocytes). Maturation of these cells occurs under the influence of a network of tissue specific protein regulators that have been given a variety of names, including growth factors, cytokines and interleukins.

Macrophages, derived from monocytes from the bloodstream, have been used as a delivery vehicle for targeting drugs and therapeutic genes to solid tumours. It has been shown that macrophages continually enter solid tumours and congregate in poorly vascularised, ischaemic sites in breast carcinomas. Moreover, the degree of ischaemia-induced necrosis in these tumours was positively correlated with the degree of intra-tumoral macrophage infiltration.

Monocytes and macrophages also infiltrate ischaemic lesions, which are a feature of other disease states including cerebral malaria, coronary heart disease and rheumatoid arthritis. Thus monocytes and macrophages are suitable host cells for use in the present invention. In particular, monocytes and/or macrophages comprising polynucleotides and/or vectors, such as viral vectors, of the invention are suitable for use in ex vivo and in vivo methods for treating diseases associated with hypoxia.

Methods for isolation of HSCs and their maintenance and differentiation in culture are known in the art (Santiago-Schwartz et al 1992; Charbord et al., 1996; Dao et al., 1997; Piacibello et al., 1997) and in WO-A-91/09938. Retroviral gene transfer into human HSCs in the general sense has been reported (Duphar and Emmons, 1994). Methods for retroviral mediated transduction of HSCs and transfer to patients are also described by Dunbar et al., 1996.

In vivo murine studies have indicated that the pretreatment of donor mice with 5-fluorouracil prior to harvest of bone marrow can improve transduction efficiencies by inducing the cycling of primitive cells and increasing the susceptibility to retroviral infection and integration. The co-culture of target cells with a retroviral producer cell line, and the use of cell lines capable of producing at least 10⁵ viral particles per ml, has also improved efficiencies (Bodine et al., 1991). Successful gene transfer into long term re-populating cells has been achieved in virtually all recipient mice with reconstitution of multiple haematopoietic lineages stably with 1-50% or more cells carrying the proviral genome (Fraser et al., 1990).

Where the invention uses a vector for delivery of an NOI to HSCs in vivo, the vector is preferably a targeted vector capable of targeting CD34⁺ HSCs.

The term “targeted vector” refers to a vector whose ability to infect/transfect a cell or to be expressed in the target cell is restricted to certain cell types within the host organism, usually cells having a common or similar phenotype. An example of a targeted vector is a targeted retroviral vector with a genetically modified envelope protein that binds to cell surface molecules found only on a limited number of cell types in the host organism. Another example of a targeted vector is one that contains promoter and/or enhancer elements which only permit expression of one or more retroviral transcripts in a proportion of the cell types of the host organism. Thus, the vector may be provided with a ligand specific for CD34, such as an antibody or an immunoglobulin-like molecule directed against CD34. On introduction into an individual to be treated such a vector will specifically transfect CD34⁺ HSCs. The vector may be administered systemically, to the peripheral circulation.

In a preferred embodiment of the invention, the target cells are cells associated with an hypoxic or ischaemic environment such as in chronic ischaemic diseases. The target cells can be cells associated with ocular neovascularization, and are most particularly, cells of the retina or retinal pigment epithelium (RPE). The target cells can also be associated with chronic ischaemic diseases such as cancer, anaemia, ischaemic retinopathies, or cardiovascular disease.

F. Therapeutic Uses

The present invention is believed to have a wide therapeutic applicability—depending on inter alia the selection of the one or more NOIs. In addition, or in the alternative, the present invention may be useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reactions and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

In a preferred embodiment of the present invention, the hypoxia-regulated lentiviral vectors are used in the treatment of ocular diseases involving an hypoxic or ischaemic component of pathology. More particularly, such ocular diseases causing severe vision loss usually result from retinal and choroidal neovascularization. There are numerous retinopathies where ischaemia is known to be of central importance to the disease pathology. These include diabetic retinopathy (PDR), age-related macular degeneration (AMD), retinopathy of prematurity, central vein occlusion, branch vein occlusion, ocular ischaemia syndrome.

In particular, polynucleotides, nucleic acid vectors, viral vectors and host cells of the present invention may be used in the treatment of tumours or ocular neovascularization. Examples of tumours that may be treated by the present invention include but are not limited to: sarcomas including osteogenic and soft tissue sarcomas, carcinomas such as breast, lung, bladder, thyroid, prostate, colon, rectum, pancreas, stomach, liver, uterine, and ovarian carcinoma, lymphomas including Hodgkin and non-Hodgkin lymphomas, neuroblastoma, melanoma, myeloma, Wilms tumour, and leukemias, including acute lymphoblastic leukemia and acute myeloblastic leukemia, gliomas and retinblastomas.

As a further example of targeting of tumours, patients with mutations in the VHL locus are known to have an increased incidence of haemagiomas and renal cell carcinomas. Furthermore, certain renal cell carcinomas have spontaneous alterations in the VHL gene. Tumour cells in these patients will have constitutively increased levels of HIF expression, which will in turn stimulate expression driven by an HRE construct regardless of the normoxia and hypoxia in the cell. Consequently, introduction of a nucleotide sequence comprising an NOI encoding a therapeutic gene product operably linked to an HRE, such as an HRE of the invention, into a cell having increased levels of HIF expression, for example as a result in a mutation at the VHL locus, may be useful in treating or prevent a condition associated with one or more mutations at the VHL locus which mutations result in inhibition of VHL function. Examples of such conditions include haemagiomas and renal cell carcinomas.

A particularly advantageous feature of the present invention is that NOIs may be expressed in hypoxic cells and not cells under normoxic conditions. Thus polynucleotides, nucleic acid vectors, viral vectors and host cells of the present invention may be used in the clinical management of a range of conditions characterised by hypoxia. Examples of conditions that are characterised by symptoms of hypoxia include retinal and choroidal neovascularization, stroke, deep vein thrombosis, pulmonary embolus and renal failure. The cell death of cardiac tissue, called myocardial infarction, is due in large part to tissue damage caused by ischemia and/or ischemia followed by reperfusion. Other examples include cerebral malaria and rheumatoid arthritis. In particular, the invention pertains to hypoxically regulated therapeutic gene expression for the long-term treatment of chronic ischaemic diseases such as cancer, ischaemic retinopathies, and cardiovascular disease.

It is especially preferred to use polynucleotides, nucleic acid vectors, viral vectors and host cells of the present invention in the clinical management of solid tumours such as ovarian tumours, in particular tumours comprising tumour cells under hypoxic conditions. Treatment may effect a slowdown in the rate of tumour growth, a cessation in the rate of tumour growth or indeed shrinkage of tumour mass without necessarily resulting in complete apoptotic/necrotic death of all malignant cells in an affected patient.

The polynucleotides, nucleic acid vectors, viral vectors and host cells of the present invention may also be used in preventative medicine. Thus the NOIs used in the invention may have a therapeutic effect via prophylaxis. For example, where an increased risk of developing cancer is diagnosed, the invention may be used to vaccinate the at-risk individual.

Suitability for prophylaxis may be based on genetic predisposition to cancer, for example cancer of the breast or ovary because of one or more mutations in a BRCA-1 gene, a BRCA-2 gene (Comelisse et al., 1996) or another relevant gene.

G. Administration

The polynucleotide, nucleic acid vectors and viral vectors of the invention may thus be used to deliver therapeutic genes to a human or animal in need of treatment.

The polynucleotide of the invention may be administered directly as a naked nucleic acid construct, preferably further comprising flanking sequences homologous to the host cell genome. Uptake of naked nucleic acid constructs by mammalian cells is enhanced by several known techniques including biolistic transformation and lipofection. Alternatively, the polynucleotide may be administered as part of a nucleic acid vector, including a plasmid vector or viral vector, preferably a lentiviral vector.

Preferably the delivery vehicle (i.e. naked nucleic acid construct or viral vector comprising the polynucleotide for example) is combined with a pharmaceutically acceptable carrier or diluent to produce a pharmaceutical composition. Thus, the present invention also provides a pharmaceutical composition for treating an individual by gene therapy, wherein the composition comprises a therapeutically effective amount of the polynucleotide, vector or viral vector of the present invention comprising one or more deliverable therapeutic and/or diagnostic NOI(s) or a viral particle produced by or obtained from same, together with a pharmaceutically acceptable carrier, diluent, excipient or adjuvant. The pharmaceutical composition may be for human or animal usage.

The choice of pharmaceutical carrier, excipient or diluent can be selected with regard to the intended route of administration and standard pharmaceutical practice. Suitable carriers and diluents include isotonic saline solutions, for example phosphate-buffered saline. The pharmaceutical compositions may comprise as—or in addition to—the carrier, excipient or diluent any suitable binder(s), lubricant(s), suspending agent(s), coating agent(s), solubilising agent(s), and other carrier agents that may aid or increase the viral entry into the target site (such as for example a lipid delivery system).

The pharmaceutical composition may be formulated for parenteral, intramuscular, intravenous, intracranial, subcutaneous, intraocular (e.g., subretinal) or transdermal administration.

Where appropriate, the pharmaceutical composition can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavemosally, intravenously, intramuscularly or subcutaneously. For parenteral administration, the composition may be best used in the form of a sterile aqueous solution, which may contain other substances, for example, enough salts or monosaccharides to make the solution isotonic with blood. For buccal or sublingual administration the composition may be administered in the form of a tablet or lozenge, which can be formulated in a conventional manner.

The pharmaceutical composition is administered in such a way that the polynucleotide/vector containing the therapeutic gene for gene therapy can be incorporated into cells at an appropriate area.

When the polynucleotide of the invention is delivered to cells by a viral vector of the invention, the amount of virus administered is in the range of from 10³ to 10¹⁰ pfu, preferably from 10⁵ to 10⁸ pfu, more preferably from 10⁶ to 10⁷ pfu. When injected, typically 1-10 μl of virus in a pharmaceutically acceptable suitable carrier or diluent is administered.

When the polynucleotide/vector is administered as a naked nucleic acid, the amount of nucleic acid administered is typically in the range of from 1 μg to 10 mg, preferably from 100 μg to 1 mg.

Where the NOI is under the control of an inducible regulatory sequence, it may only be necessary to induce gene expression for the duration of the treatment. Once the condition has been treated, the inducer is removed and expression of the NOI is stopped. This will clearly have clinical advantages. Such a system may, for example, involve administering the antibiotic tetracycline, to activate gene expression via its effect on the tet repressor/VP16 fusion protein.

The use of tissue-specific promoters will be of assistance in the treatment of disease using the polynucleotides/vectors of the invention. For example, several neurological disorders are due to aberrant expression of particular gene products in only a small subset of cells. It will be advantageous to be able express therapeutic genes in only the relevant affected cell types, especially where such genes are toxic when expressed in other cell types.

Modified HSCs of the invention are administered to a patient or an at-risk individual in a suitable formulation. The formulation may include an isotonic saline solution, a buffered saline solution or a tissue-culture medium. The cells are administered by bolus injection or by infusion intravenously or directly to the site of a tumour or to the bone marrow at a concentration of for example between approximately 10⁶ and of the order of 10¹² cells/dose, preferably at least 10⁸ or 10¹⁰ cells per dose.

The individual may first be treated to deplete the bone marrow of stem cells or may be treated with one or more cytokines such as G-CSF to increase the mobilisation of stem cells into the peripheral blood or one or more cytokines to enhance repopulation of bone marrow. Combinations of such treatments are also envisaged. The treatments of the invention may also be combined with currently available anti-cancer therapies.

In the event that the vector used for stem cell engineering encodes a pro-drug activating enzyme, the individual suffering from cancer is additionally treated with the corresponding pro-drug, administered using an appropriate regimen according to principles known in the art.

Where the HSCs are removed from the individual to be treated, and are transfected or transduced with the vector in vitro, the cells are generally expanded in culture prior to and after introduction of the NOI or NOIs. When cultured in vitro under appropriate conditions or when appropriate signals are received in vivo, HSC have the capacity to differentiate into, among other cell types, endothelial cells, myeloid cells, dendritic cells and immune effector cells such as neutrophils, lymphocytes, mononuclear phagocytes and NK cells.

This involves the use of tissue culture methods that are known in the art and include exposure to cytokines and/or growth factors for the maintenance of HSCs (Santiago-Schwartz et al., 1992; Charbord et al., 1996; Dao et al., 1997; Piacibello et al., 1997). Agents that induce the differentiation of the HSCs may also be added.

In addition to responding to hypoxia the HRE elements are known to respond to chemical inducers that mimic hypoxia. Two of these are known, these are cobalt chloride and desferrioxamine (Meliillo et al., 1996; Wang and Semenza 1993b). Thus, the products of the invention may also be used to treat a disorder where compounds that mimic hypoxia are administered, such as the chemical activator desferrioxamine or analogous chemicals used to treat neuroblastoma (Blatt, 1994), beta thalassemia (Giardina and Grady, 1995), Alzheimers disease (Crapper et al., 1991), VEGF deficiency (Beerrepoot et al., 1996), Erythropoetin deficiency (Wang and Semenza, 1993b) and for enhancement of tumour chemotherapy (Voest et al., 1993). The products of the invention may be administered concomitantly, sequentially or separately with the compounds that mimic hypoxia.

The routes of administration and dosages described are intended only as a guide since a skilled practitioner will be able to determine readily the optimum route of administration and dosage for any particular patient and condition.

In accordance with the invention, standard molecular biology techniques may be used which are within the level of skill in the art. Such techniques are fully described in the literature. See for example; Sambrook et al (1989) Molecular Cloning; a laboratory manual; Ausubel et al., Current Protocols in Molecular Biology (1999), John Wiley & Sons, Inc.; Hames and Glover (1985-1997) DNA Cloning: a practical approach, Volumes I-IV (second edition); Methods for the engineering of immunoglobulin genes are given in McCafferty et al., 1996, “Antibody Engineering: A Practical Approach”.

It should be appreciated that features from various sections, aspects and embodiments of the invention as described above are generally equally applicable to other sections, aspects and embodiments mutatis mutandis.

The invention will now be further described by way of Examples, which are meant to serve to assist one of ordinary skill in the art in carrying out the invention and are not intended in any way to limit the scope of the invention.

### EXAMPLES

### Example 1

### Plasmid Construction

Synthetic oligonucleotides are synthesised encompassing hypoxia response element (HRE) sequences and cloned as BglII/BamH1 fragments into the BamH1 site of the pGL3 promoter plasmid (Promega; Genbank accession no U47298). PGK sequences are synthesised as Xba1/Nhe1 fragments and cloned into the Nhe1 site of this vector pGL3 is an enhancerless expression plasmid with a minimal SV40 promoter upstream of a luciferase coding sequence. Insertion of the HRE at this site places it upstream of the minimal SV40 promoter.

OBHRE1

Trimer encompassing −307/−290 sequence of murine PGK in the natural orientation (Firth et al., 1995) linked to the SV40 promoter (italicised) (SEQ ID NO:30).

```
GCTAGAGTCGTGCAGGACGTGACATCTAGTGTCGTGCAGGACGTGACA
Nhel HRE HRE
TCTAGTGTCGTGCAGGACGTGACAGCTAGCCCGGGCTCGAGATCTGCG
                              HRE Xbal
ATCTGCATCTCAATTAGTCAGCAACCATAGTCCCGCCCCTAACTCCGCC
CATCCCGCCCCTAACTCCGCCCAGTTCCGCCCATTCTCCGCCCCATCG
CTGACTAATTTTTTTTATTTATGCAGAGGCCGAGGCCGCCTCGGCCTCTG
                                                                                startstart
```

Lentiviral Vector Configurations

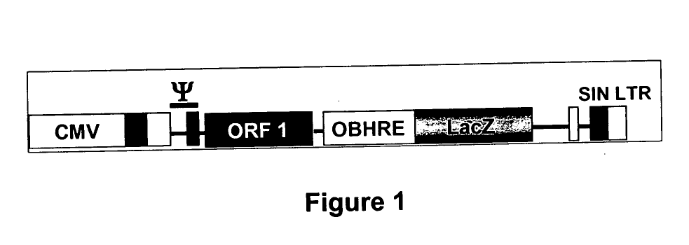

The OBHRE hypoxia responsive promoter has been integrated into a minimal EIAV LentiVector platform, shown schematically in FIG. 1. This vector is based on the pONY8.4 series of vectors. The pONY8 series of vectors are derived from the pONY4 series of vectors and the pONY2 series of vectors. The pONY2.1 is one of the vector derivatives from the pONY series of EIAV vectors. pONY2.1 includes a partial deletion of gag, pONY4 further includes a deletion to make the tat, S2, env, and rev genes non-functional, and pONY8 includes further deletions in the gag, tat, S2 env, and rev genes. Details relating to the pONY series of EIAV vectors are found in GB9727135.7 and WO99/32646. Details are also found in Kim et al. J. Virol. (1998) 72:811-6; Mitrophanous et al. Gene Ther. (1999) 6:1808-18; and Mazarakis et al. Hum. Mol. Gen. (2001) 10(19): 2109-21. The specific configuration of the EIAV vectors can be optimized for obtaining a low basal/high inducible expression profile within the vector platform. Firstly, the OBHRE LacZ is present as an internal expression cassette. Upstream of the OBHRE LacZ cassette is an open reading frame (ORF1), which can be any ORF such as (but not limited to) neomycin, HPRT, and green fluorescent protein (GFP). There is limited/no expression of the upstream ORF in the transduced target cells since the 3′LTR is self-inactivating (SIN LTR)—i.e. all enhancer sequences have been removed. The ORF is important to obtain low basal activity of the OBHRE promoter in non-inducing conditions, possibly by minimising any promoter interference in the target cells.

### Example 2

### Transduction of Human Retinal Pigment Epithelial Cells with EIAV-OBHRE

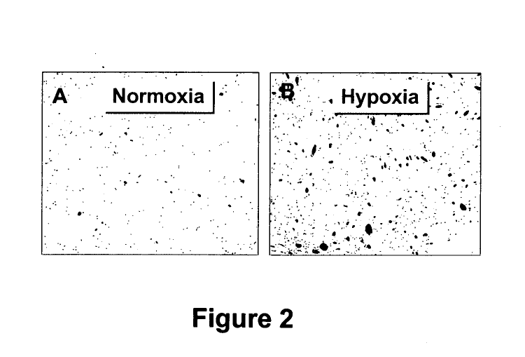

The EIAV-OBHRE recombinant LentiVector shows hypoxically regulated LacZ expression in transduced human retinal pigment epithelial cells, the ARPE-19 cell line (FIG. 2). ARPE-19 cells were transduced with the EIAV-OBHRE recombinant LentiVector at an MOI of 20. After 24 hours, the cells were either maintained in 20% oxygen or were exposed to 0.1% oxygen for 16 hours. Finally the cells were X-gal stained to reveal the level of LacZ expression.

In the case of gene therapy for AMD and/or PDR it would be ideal to target cells of the retinal pigment epithelium (RPE) or those of the outer retina such as the photoreceptor (PR) cell layer that are specifically affected by ocular neovascularisation.

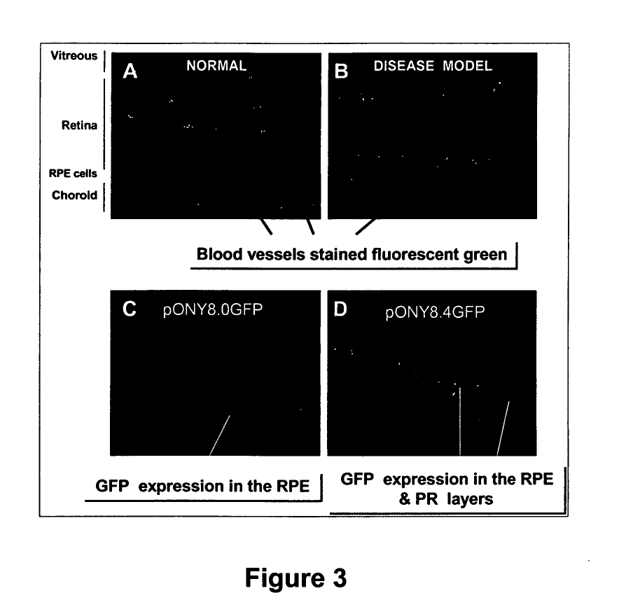

The gene transfer characteristics of a non-primate EIAV-based lentivirus vector following sub-retinal gene delivery are shown in FIG. 3. The LentiVectors can be pseudotyped with either VSV G or Rabies G envelopes to obtain similar (or analogous) expression profiles. In addition, other envelope pseudotypes can be incorporated during the production process that may impact biodistribution; these may include, for example, Ross River, Mokola, and Ebola envelopes.

Depending on the inherent nature of the EIAV lentivirus genome, it may be possible to limit gene expression specifically to the RPE layer, using the pONY8.0 vector series or to obtain gene expression in both the RPE and PR cell layers, using the pONY8.4 up to pONY8.9 series of vectors that include the Woodchuck hepatitis virus post-transcriptional regulatory element (WPRE). Addition of the WPRE can reduce the baseline normoxia expression, therefore increasing the overall fold induction in some target cells. Furthermore, the incorporation of cell-specific promoters may further target gene expression to particular cellular compartments. Examples of this would include the promoter and enhancer sequences derived from particular genes that show restricted/cell-specific expression in the retina/ocular compartment. In the context of targeting the RPE, a tissue specific promoter such as the VMD2 promoter is preferred.

Sub-retinal delivery of the EIAV LentiVector leads to strong transgene expression that persists for at least 6 months. Therefore, the EIAV vector is an ideal vehicle for delivering a long-term therapeutic angiostatic molecule to the relevant location in ocular neovascular disorders such as AMD and PDR.

### Example 3

### Use of ELAV-OBHRE in a Murine Model of Choroidal Neovascularsation

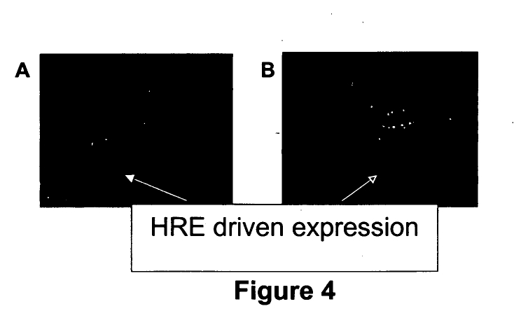

The EIAV-OBHRE recombinant LentiVector has been evaluated in the murine model of laser induced choroidal neovascularisation. In this study mice received a subretinal injection of LentiVector on day 0 (a dose of 3.2×10e6 TU/eye in a total volume of 4 ul). Laser injury to Bruch's membrane was carried out 7 days later and the eyes were enucleated for histological analysis on day 14. OBHRE mediated LacZ expression was observed in localised regions of the laser treated retinas; however, LacZ expression was not detected in the unlasered retina (FIG. 4).

The utility of the OBHRE promoter to hypoxically-regulate transgene expression in experimental retinal and choroidal neovascularisation has been further demonstrated using a recombinant adeno-associated virus vector, rAAV.HRE.GFP (Bainbridge et al 2003 Gene Ther.). In murine ischaemia-induced retinal neovascularisation, intravitreal delivery of the rAAV.HRE.GFP vector led to expression specifically at sites of vascular closure only during the period of active neovascularisation. Similarly, in the murine laser-induced CNV model, subretinal delivery of the same vector resulted in reporter gene expression at sites of active neovascularisation but not elsewhere. These data not only outline the spatial restriction of gene expression imposed by the OBHRE promoter but also the temporal restriction such that reporter gene expression ceases upon disease resolution in both of these models.

### Example 4

### Use of Hypoxia-Regulated LentiVectors for Delivery of Angiostatic Factors

Therapeutic Strategy

An effective gene therapy strategy for diseases involving ocular neovascularisation requires the following:

An angiostatic molecule/gene that targets neovascularisation, VEGF, is the principal mediator of ocular neovascularisation, and is therefore of significant importance in the development of the disease and an ideal target for therapeutic intervention.

A gene encoding a molecule that has a potent effect on reversing established choroidal neovascular lesions, as well as preventing the de novo formation of lesions would be a useful candidate gene.

A gene encoding a molecule that has a potent effect on addressing the excessive vascular permeability arising in such ocular pathologies would be a useful gene candidate.

A vector system that is non-immunogenic, that can target non-dividing cells at the relevant ocular location, that has long-term expression kinetics, and that is ideally regulated by disease pathology and disease resolution is desirable.

Candidate Genes

There are several examples of candidate therapeutic genes that encode active angiostatic molecules. These include, but are not limited to, the molecules discussed below.

Endostatin is an angiogenesis inhibitor produced by hemangioendothelioma, that was first identified by O'Reilly et al. (1997). Endostatin is a 20 kD C-terminal fragment of collagen XVIII that specifically inhibits endothelial proliferation, and potently inhibits angiogenesis and tumor growth. In fact, primary tumors have been shown to regress to dormant microscopic lesions following the administration of recombinant endostatin (O'Reilly et al., 1997). Endostatin is reported to inhibit angiogenesis by a number of mechanisms including: binding to the heparin sulphate proteoglycans involved in growth factor signalling (Zetter, 1998), inhibiting endothelial invasion via MMP inhibition, inhibiting endothelial cell:matrix adhesion via interactions with α5.αv integrins, physically interacting with VEGFR2, impacting on cytoskeletal rearrangement via interacting with cytoskeletal proteins including tubulin. In addition endostatin can significantly ablate the vascular permeability observed in pathological angiogenesis.

Angiostatin is an internal fragment of plasminogen, comprising the first four kringle structures (although the fourth kringle domain has since been shown to be dispensable for the angiostatic activity) and is one of the most potent endogenous angiogenesis inhibitors described to date. For example, it has been shown that systemic administration of angiostatin efficiently suppresses malignant glioma growth in vivo (Kirsch et al., 1998). Angiostatin has also been combined with conventional radiotherapy resulting in increased tumor eradication without increasing toxic effects in vivo (Mauceri et al., 1998). Other studies have demonstrated that retroviral and adenoviral mediated gene transfer of angiostatin cDNA resulted in the inhibition of endothelial cell growth in vitro and angiogenesis in vivo. The inhibition of tumor-induced angiogenesis produced an increase in tumor cell death (Tanaka et al., 1998). Gene transfer of a cDNA coding for mouse angiostatin into murine T241 fibrosarcoma cells has been shown to suppress primary and metastatic tumor growth in vivo (Cao et al., 1998).

VEGF Receptor 1/fms-like tyrosine kinase 1 receptor (VEGFR1/solFLT1) is a membrane-bound receptor of VEGF. It has been shown that a soluble fragment of FLT-1 (sFLT-1) has angiostatic properties by way of its antagonist activity against VEGF, probably because it binds VEGF but also because it binds and blocks the external domain of the membrane-bound FLT-1 (R L Kendall & K A Thomas 1993, C K Goldman et al. 1998). This candidate gene has been successfully evaluated in animal models of AMD and PDR.

Pigment Epithelium-Derived Factor (PEDF) was discovered because of its neurotrophic activity in the fetal retina (Steele et al 1993). It is produced by retinal cells to prevent vascularization of eye cornea and vitreous, and has been shown to be a potent inhibitor of angiogenesis (King et al 2000, Dawson et al 1999).

Other therapeutic candidates that directly interfere with the VEGF angiogenic factor are candidates such as aptamers, antibodies, etc.

Endostatin & Angiostatin

Endostatin and angiostatin are particularly attractive candidates for an ocular gene therapy strategy, largely due to their mode of action against neovessel development.

Angiogenesis is a multi-step process that includes endothelial cell proliferation, migration, basement membrane degradation, new lumen organization, and permeability of the new vessels. The development of new blood vessels is controlled by a local balance between stimulators and inhibitors of new vessel growth. Unlike some candidate angiogenic approaches that target a single angiogeneic factor such as VEGF, angiogenesis inhibitors such as angiostatin and endostatin have been shown to modulate angiogenesis at various stages of the angiogenic process including proliferation, migration, tubule formation, permeability. Moreover, it has been reported that the action of angiostatin in the eye is specific to pathological angiogenesis and not developmental angiogenesis (Drixler et al IOVS, 2001). Angiostatin is not normally found in the human vitreous, however production of angiostatin correlates with retinal photocoagulation treatment in patients with diabetic retinopathy indicating that this molecule is produced in response to damage to the retina in an effort to prevent pathological neovessel formation (Spranger et al, Diabet, 2000).

There is now a significant body of published data, demonstrating the efficacy of endostatin or angiostatin in different virus vector platforms in various ocular models. For example, a lentiviral vector based on BIV expressing endostatin has been shown to reduce VEGF-induced retinal vascular permeability, neovascularisation and retinal detachment (Takahashi et al FASEB, 2003). In addition, a recombinant adeno associated virus (AAV) expressing endostatin has been shown to inhibit neovascularisation in a retinopathy of prematurity (ROP) murine model (Auricchio et al Mol Ther 2003). Further, an adenovirus vector expressing endostatin has been shown to suppress rat CNV (Lai et al IOVS 2001). The story is similar for angiostatin. AAV vectors expressing angiostatin have been shown to suppress murine CNV and reduce retinal neovascularisation (Lai et al IOVS 2001, Raisler et al PNAS, 2002). In addition, a HIV vector expressing angiostatin inhibited murine CNV (Igarashi et al Gene Ther, 2003)

Interestingly, the effects of angiostatin and endostatin on angiogenesis seem to be synergistic. In a paper published in Nature (Boehm et al.,1997, Nature 390: 404-407), it was shown that treatment of mice with Lewis lung carcinomas with the combination of endostatin and angiostatin proteins induced the complete regression of the tumours, and that mice remained healthy for the rest of their life. This effect was obtained only after one cycle (25 days) of endostatin and angiostatin treatment, whereas endostatin alone required 6 cycles to induce tumor dormancy. In another study published in Science (Bergers et al., 1999, Science 284: 808-812), the superior antitumoural effect of the combination of endostatin and angiostatin proteins in a mouse model for pancreatic islet carcinoma. The endostatin and angiostatin combination resulted in a significant regression of the tumours, whereas endostatin or angiostatin alone had no effect.

Therefore, it is possible that the potency of a gene therapy vector could be improved by delivering both genes in equimolar proportions. The genes could be delivered, for example as a fusion protein. Recombinant fusion proteins have been evaluated in tumours models, with a high degree of potency compared to the single proteins. Alternatively, the genes could be delivered bicistronically within the same LentiVector, such that they are separated by an internal ribosome entry site (IRES), such as that from the EMCV virus. A third option would be to deliver the genes in two different vectors. It is also important to consider regulation of gene expression. In this context, an EIAV LentiVector could be developed where expression is regulated by a hypoxically responsive promoter. Therefore, expression is limited temporally and spatially to diseased regions of tissue.

4.1 Construction of EIAV Vectors Expressing Endostatin and/or Angiostatin via Either a CMV or HRE Promoter

Codon Optimisation

Codon bias is observed in all species, and the use of selective codons in genes often correlates with gene expression efficiency. It is generally understood that taxonomically-close organisms, such as E. coli and Salmonella, for example, use similar codons for their protein synthesis, whereas, in taxonomically-distant organisms, such as E. coli and mammals codon usage is very different. Thus, differences between codon usage in a heterologous gene and the host organism may affect expression.

The greatest deviation from random codon usage in an organism occurs in the most highly expressed genes, as a result of selection for codons that maximise translational efficiency. Minor tRNA species are avoided in highly expressed genes. Therefore, manipulating a gene with a relatively poor codon usage to one with an optimised biased codon usage can dramatically improve expression levels.

In addition to altering the codon usage of the desired gene to that of highly expressed genes, the process of codon optimisation enables the scientist to engineer a cDNA sequence that avoids, where possible, the following regions that could impact on recombinant virus vector production as well as protein expression:

Regions of very high (>80%) or very low GC (<30%) content are avoided where possible.

Internal TATA-boxes, chi sites and ribosome entry sites are avoided.

AT rich and GC rich sequences stretches are removed where possible.

Repeated sequences, RNA secondary structures, (cyptic) splice donor and acceptor sites and branch points are removed where it is possible.

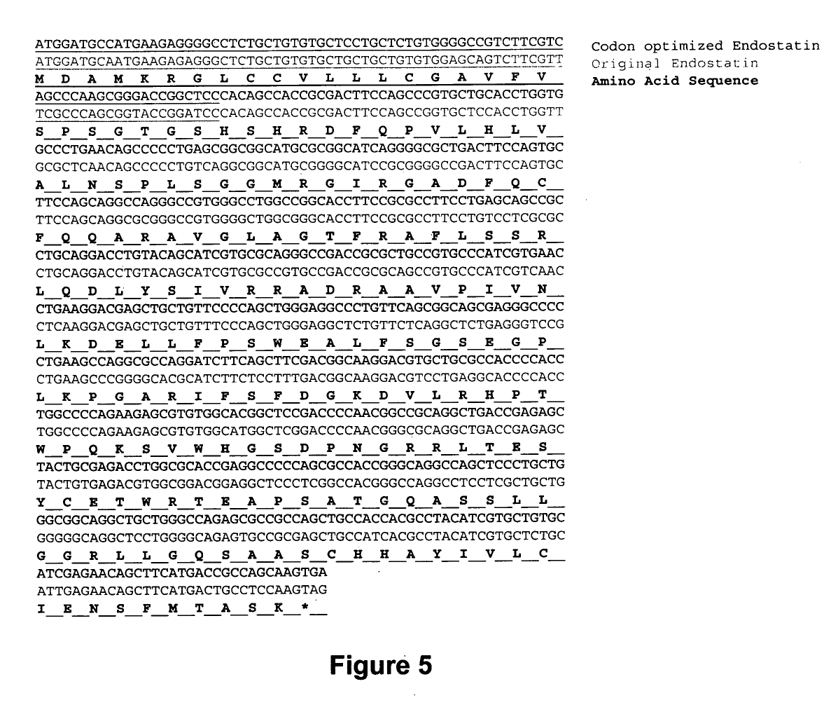

The codon optimised human endostatin sequence is shown in FIG. 5, aligned against the original sequence. Table 2 highlights the major changes during the optimisation process.

TABLE 2
Codon Usage Comparison for Human Endostatin

| | | | Mammalian | |
|---|---|---|---|---|
| | | Native | highly | Codon |
| Amino | | Sequence | expressed | optimized |
| Acid | Codon | Freq/% | genes | Freq/% |
| ALA | GCA | (4) 20 | 13 | (0) 0 |
| | GCC | (10) 50 | 53 | (18) 90 |
| | GCG | (4) 20 | 17 | (0) 0 |
| | GCT | (2) 10 | 17 | (2) 10 |
| ARG | AGA | (1) 6 | 10 | (0) 0 |
| | AGG | (3) 19 | 18 | (7) 43 |
| | CGA | (0) 0 | 6 | (0) 0 |
| | CGC | (8) 50 | 37 | (9) 57 |
| | CGG | (3) 19 | 21 | (0) 0 |
| | CGT | (1) 6 | 7 | (0) 0 |
| ASN | AAC | (4) 100 | 78 | (4) 100 |
| | AAT | (0) 0 | 22 | (0) 0 |
| ASP | GAC | (8) 88 | 75 | (8) 88 |
| | GAT | (1) 12 | 25 | (1) 12 |
| CYS | TGC | (4) 57 | 68 | (5) 71 |
| | TGT | (3) 43 | 32 | (2) 29 |
| GLN | CAA | (0) 0 | 12 | (0) 0 |
| | CAG | (8) 100 | 88 | (8) 100 |
| GLU | GAA | (0) 0 | 25 | (0) 0 |
| | GAG | (7) 100 | 75 | (7) 100 |
| GLY | GGA | (2) 10 | 14 | (0) 0 |
| | GGC | (9) 45 | 50 | (17) 85 |
| | GGG | (7) 35 | 24 | (3) 15 |
| | GGT | (2) 10 | 12 | (0) 0 |
| HIS | CAC | (5) 71 | 79 | (7) 100 |
| | CAT | (2) 29 | 21 | (0) 0 |
| ILE | ATA | (0) 0 | 5 | (0) 0 |
| | ATC | (5) 83 | 18 | (6) 100 |
| | ATT | (1) 17 | 77 | (0) 0 |
| LEU | CTA | (0) 0 | 3 | (0) 0 |
| | CTC | (6) 25 | 26 | (3) 12 |
| | CTG | (18) 75 | 58 | (21) 88 |
| | CTT | (0) 0 | 5 | (0) 0 |
| | TTA | (0) 0 | 2 | (0) 0 |
| | TTG | (0) 0 | 6 | (0) 0 |
| LYS | AAA | (0) 0 | 18 | (0) 0 |
| | AAG | (6) 100 | 82 | (6) 100 |
| PHE | TTC | (9) 82 | 80 | (11) 100 |
| | TTT | (2) 18 | 20 | (0) 0 |
| PRO | CCA | (0) 0 | 16 | (2) 18 |
| | CCC | (9) 82 | 48 | (9) 88 |
| | CCG | (2) 18 | 17 | (0) 0 |
| | CCT | (0) 0 | 19 | (0) 0 |
| SER | AGC | (9) 39 | 34 | (20) 87 |
| | AGT | (1) 4 | 10 | (0) 0 |
| | TCA | (2) 9 | 5 | (0) 0 |
| | TCC | (5) 22 | 28 | (3) 13 |
| | TCG | (5) 22 | 9 | (0) 0 |
| | TCT | (1) 4 | 13 | (0) 0 |
| THR | ACA | (0) 0 | 14 | (0) 0 |
| | ACC | (4) 50 | 57 | (8) 100 |
| | ACG | (3) 37 | 15 | (0) 0 |
| | ACT | (1) 23 | 14 | (0) 0 |
| TYR | TAC | (3) 100 | 74 | (3) 100 |
| | TAT | (0) 0 | 26 | (0) 0 |
| VAL | GTA | (0) 0 | 5 | (0) 0 |
| | GTC | (3) 25 | 25 | (2) 17 |
| | GTG | (7) 58 | 64 | (10) 83 |
| | GTT | (2) 17 | 7 | (0) 0 |

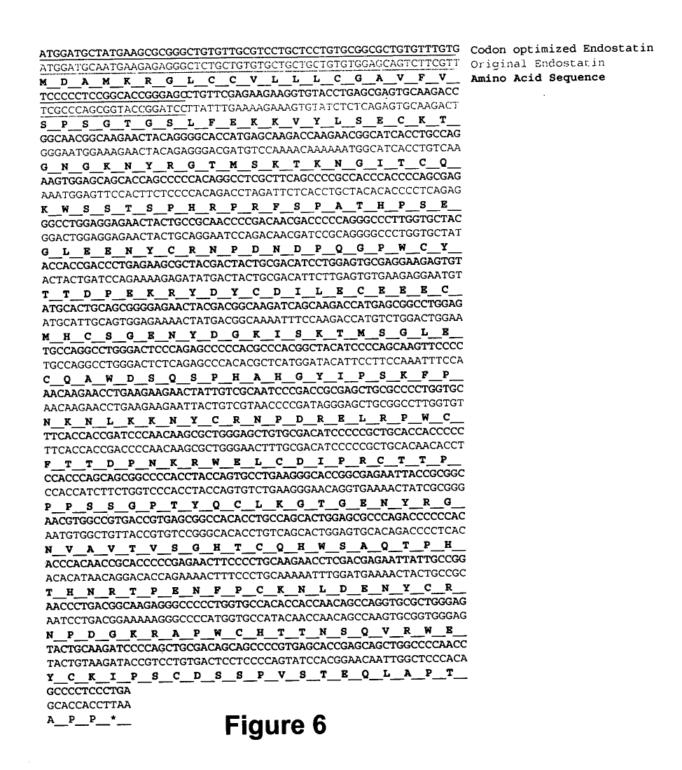

The codon optimised human angiostatin sequence is shown in FIG. 6, aligned against the original sequence. Table 3 highlights the major changes during the optimisation process.

TABLE 3
Codon Usage Comparison for Human Angiostatin

| | | | Mammalian | |
|---|---|---|---|---|
| | | Native | highly | Codon |
| Amino | | Sequence | expressed | optimized |
| Acid | Codon | Freq/% | genes | Freq/% |
| ALA | GCA | (4) 40 | 13 | (0) 0 |
| | GCC | (2) 20 | 53 | (8) 80 |
| | GCG | (0) 0 | 17 | (0) 0 |
| | GCT | (4) 40 | 17 | (2) 20 |
| ARG | AGA | (5) 35 | 10 | (0) 0 |
| | AGG | (4) 29 | 18 | (3) 19 |
| | CGA | (0) 0 | 6 | (0) 0 |
| | CGC | (4) 29 | 37 | (12) 75 |
| | CGG | (0) 0 | 21 | (1) 6 |
| | CGT | (1) 7 | 7 | (0) 0 |
| ASN | AAC | (13) 65 | 78 | (17) 85 |
| | AAT | (7) 35 | 22 | (3) 15 |
| ASP | GAC | (9) 64 | 75 | (12) 86 |
| | GAT | (5) 36 | 25 | (2) 14 |
| CYS | TGC | (12) 52 | 68 | (20) 87 |
| | TGT | (11) 48 | 32 | (3) 13 |
| GLN | CAA | (3) 33 | 12 | (0) 0 |
| | CAG | (6) 67 | 88 | (9) 100 |
| GLU | GAA | (11) 58 | 25 | (1) 5 |
| | GAG | (8) 42 | 75 | (18) 95 |
| GLY | GGA | (9) 45 | 14 | (0) 0 |
| | GGC | (2) 10 | 50 | (17) 85 |
| | GGG | (6) 30 | 24 | (3) 15 |
| | GGT | (3) 15 | 12 | (0) 0 |
| HIS | CAC | (6) 60 | 79 | (10) 100 |
| | CAT | (4) 40 | 21 | (0) 0 |
| ILE | ATA | (1) 17 | 5 | (0) 0 |
| | ATC | (2) 33 | 18 | (6) 100 |
| | ATT | (3) 50 | 77 | (0) 0 |
| LEU | CTA | (0) 0 | 3 | (0) 0 |
| | CTC | (2) 13 | 26 | (2) 13 |
| | CTG | (8) 54 | 58 | (13) 87 |
| | CTT | (2) 13 | 5 | (0) 0 |
| | TTA | (1) 7 | 2 | (0) 0 |
| | TTG | (2) 13 | 6 | (0) 0 |
| LYS | AAA | (8) 40 | 18 | (0) 0 |
| | AAG | (12) 60 | 82 | (20) 100 |
| PHE | TTC | (3) 50 | 80 | (5) 83 |
| | TTT | (3) 50 | 20 | (1) 17 |
| PRO | CCA | (10) 33 | 16 | (2) 7 |
| | CCC | (10) 33 | 48 | (23) 76 |
| | CCG | (2) 6 | 17 | (0) 0 |
| | CCT | (8) 28 | 19 | (5) 17 |
| SER | AGC | (3) 12 | 34 | (22) 88 |
| | AGT | (3) 12 | 10 | (0) 0 |
| | TCA | (3) 12 | 5 | (0) 0 |
| | TCC | (10) 40 | 28 | (3) 12 |
| | TCG | (1) 4 | 9 | (0) 0 |
| | TCT | (5) 20 | 13 | (0) 0 |
| THR | ACA | (9) 36 | 14 | (0) 0 |
| | ACC | (10) 40 | 57 | (25) 100 |
| | ACG | (2) 8 | 15 | (0) 0 |
| | ACT | (4) 16 | 14 | (0) 0 |
| TYR | TAC | (8) 61 | 74 | (11) 84 |
| | TAT | (5) 39 | 26 | (2) 16 |
| VAL | GTA | (1) 11 | 5 | (0) 0 |
| | GTC | (1) 11 | 25 | (1) 11 |
| | GTG | (5) 56 | 64 | (8) 89 |
| | GTT | (2) 22 | 7 | (0) 0 |

4.2 Construction of an EIAV Endostatin IRES Angiostatin Genome

The following describes the construction of an EIAV Endostatin IRES Angiostatin Genome, based on the pONY9.1 genome.

The key features of the pONYK9.1 genome plasmid are that it is a kanamycin resistant plasmid; it contains the WPRE element—no cPPT; it has an internal, bicistronic expression cassette including both the human endostatin and angiostatin genes separated by the EMCV IRES; and it includes an upstream open reading frame that is the HPRT cDNA. This is the hypoxanthine-guanine phosphoribosyl transferase (HPRT), which is a ubiquitous enzyme found in virtually all mammalian cells and is involved in nucleoside metabolism and purine salvage.

The cloning steps are as follows:

Step 1:

The human angiostatin sequence (Table 3) is synthesised with a 3′ flanking sequences that include PacI (TTAATTAA) and BamHI (GGATCC) restriction sites:

3′ Flank—TGA—GTTAATTAAGGATCC (SEQ ID NO:31)

The ATG of the angiostatin is encompassed in an NcoI restriction site (CCATGG).

The angiostatin cDNA is cloned as an NcoI-BamHI fragment into the pSP72-IRES plasmid NcoI-BamHI sites. The pSP72—IRES plasmid contains the EMCV ires and using the NcoI site at the end of the ires will ensure that the angiostatin ATG is optimally positioned as the 11^{th} ATG of the ires to ensure maximal translation. The resulting plasmid is called pSP72-IRESAngio

Step 2:

The human endostatin sequence (Table 2) is synthesised with 5′ and 3′ flanking sequences that include XhoI (CTCGAG) and NruI (TCGCGA) at the 5′ and an XbaI (TCTAGA) in the 3′ flank.

| | |
|---|---|
| 5′ Flank-CTCGAGTCGCGAGCCACC-ATG | (SEQ ID NO:32) |
| 3′ Flank-TGA-TCTAGA | |

The endostatin cDNA is cloned as a XhoI and XbaI into the pSP72—IRESAngio plasmid XhoI XbaI sites so that the endostatin is upstream of the IRES. The resulting plasmid is called pSP72-EiresA.

Step 3:

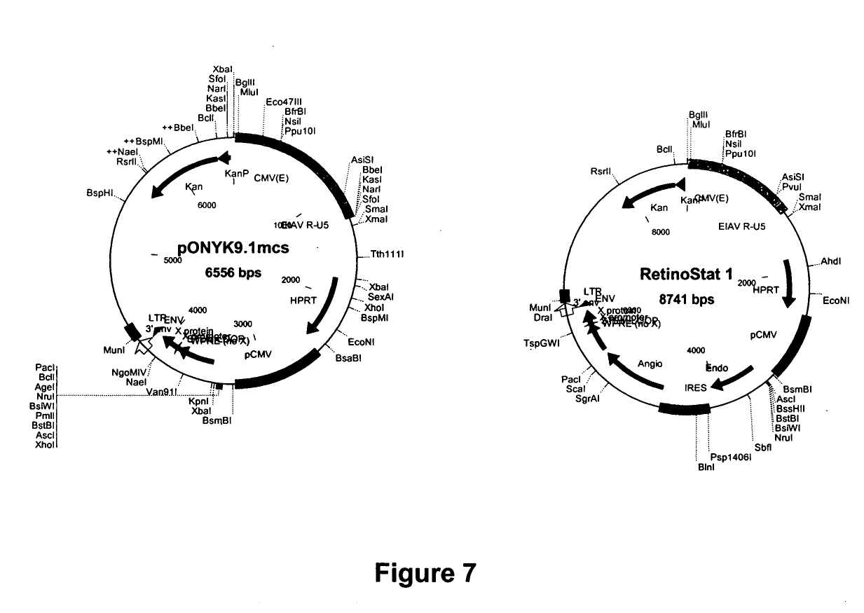

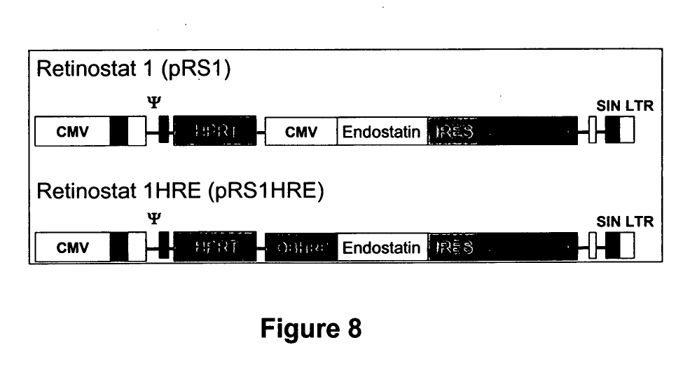

The next step is to clone the endostatin IRES angiostatin fragment from pSP72-EiresA as an NruI-PacI fragment into the NruI-PacI sites of the pONYK9.1mcs EIAV genome shown in FIG. 7. This will create the pRS1 constitutive genome shown in FIG. 8.

Step 4:

The hypoxia regulated EIAV vector is generated from the pRS1 genome. An oligonucleotide is prepared:

Stat1 primer has a SalI compatible end (5′) and a NruI (3′) compatible end and has NheI and SalI sites in the middle (NB the 5′ SalI site is destroyed upon cloning in to a SalI site in the genome:

Sall Nhel Sall Nrul
TCGA-GCTAGC-GTCGAC-TCG
CGATCG-CAGCTG-AGC

The oligonucleotide is cloned in to the SalI-NruI digested RS1 genome plasmid. Next, OBHRE promoter from the pONY8.4NHLacZ plasmid is removed as an NheI-XhoI fragment, and cloned upstream of the endostatin in the pRS1 genome plasmid to create pRS1HRE.

4.3 Use of Hypoxia-Regulated Lentiviral Vectors Expressing Angiostatic Genes for Non-Ocular Applications

An HRE-regulated angiostatic lentiviral vector would be appropriate for other disease settings where angiogenesis is known to be involved in disease progression, such as cancer and arthritis.

HIF-mediated HRE activation is a defining feature of tumours. This not only occurs as a result of hypoxia/ischaemia secondary to compromised vascularisation, but also can occur as a consequence of genomic changes, for example, in genes such as p53 and PTEN.

Hypoxia-mediated angiogenesis is known to play a part in the pathogenesis of inflammatory diseases such as rheumatoid arthritis. For example, intra-articular application of the angiostatic molecule angiostatin reduces the severity of collagen-induced arthritis in mice and the expression of HIF-1alpha in myeloid cells is important for the initiation of the inflammatory infiltrate in rheumatoid arthritis.

Both of these diseases are potential targets of an angiostatic gene therapy product that is regulated by a hypoxia responsive promoter (e.g. the RetinoStat vector series).

4.4. Recombinant EIAV Vector Production and Validation

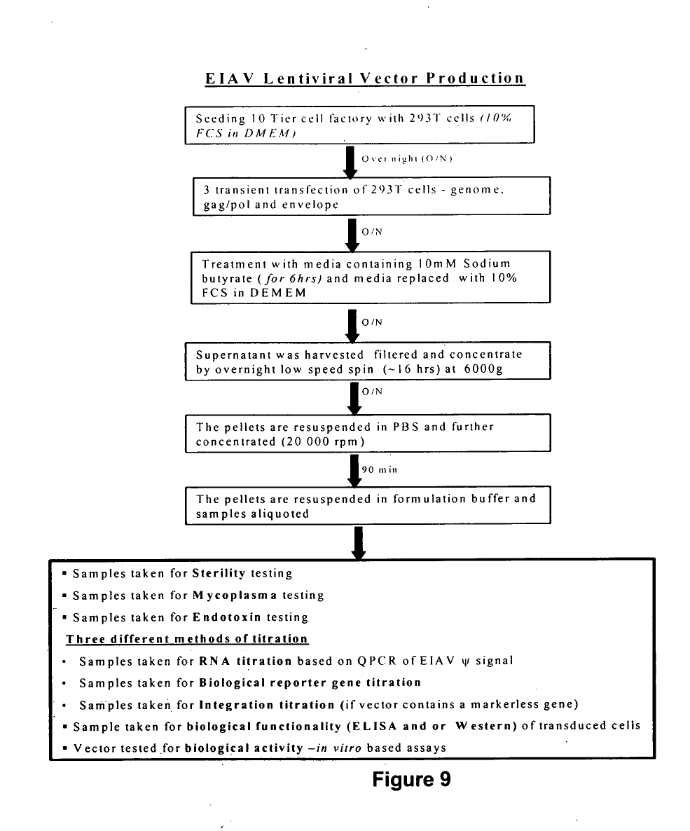

The production of a recombinant EIAV vector is shown schematically in FIG. 9. The final vector product is initially tested for sterility, mycoplasma and endotoxin. The titre of each recombinant virus is determined using quantitative RTPCR (Q RTPCR) using the EIAV packaging signal as a template for the PCR. If the recombinant virus contains a detectable marker gene such as β-galactosidase (LacZ) or GFP, then the virus can also be biologically titered on a cell line (such as D17 or 293T). In the case of the RetinoStat 1 and Retinostat HRE, lentiviral vectors that do contain any marker genes these can be titered for integration via passaging the viral vector through an appropriate cell line such as 293T.

The biological activity and functionality of the RetinoStat 1 and Retinostat HRE viral vectors can be determined by harvesting media supernatant from transduced cells (such as D17 or 293T) that contain the expressed and secreted protein products. The supernatant can then be tested for the presence of the protein products using a Western blotting or ELISA based assay. In the case of the RetinoStat HRE virus, the protein products are only expressed when the transduced cells are exposed to hypoxic conditions. Therefore, the protocol for harvesting supernatant from these cells would be to transduce the target cells on day 0. The media is replaced on day 1 and the cells are exposed to 0.1% oxygen conditions overnight on day 2. The media is harvested on days 4 and 5 for analysis in different in vitro assays.

The biological functionality of the protein products, endostatin and angiostation can be determined using any one of many in vitro angiogenesis based assays. Such assays include (but are not limited to):

Human umbilical vein endothelial cell (HUVEC) proliferation assay

HUVEC migration assay

HUVEC matrigel assay

HUVEC/HDAF (human dermal fibroblast) tubulogenesis assay

Rat aortic ring based migration/proliferation assay

4.5 In Vivo Studies to Evaluate Angiostatic HRE-Regulated or Constitutive Lentiviral Vectors

Initially, candidate lentiviral vectors are evaluated in well-established models of age-related macular degeneration, based on laser rupture of Bruch's membrane, which leads to choroidal neovascularisation (CNV) that extends into the retinal layers. The models may be murine, primate or porcine.

The vector is administered subretinally at a dose appropriate for the size of the animal model. For example, a total volume of 4 μl is suitable for a single murine eye, and a total of 100 μl is suitable for the porcine eye, which is most similar in size to the human eye. The vector is either delivered before laser rupture or prior to laser injury, as is more relevant to the real disease state. Efficacy may be determined in several ways including:

Causing a reduction in laser induced lesion size over time, as determined by perfusion of a fluorescent dye.

Causing a reduction in permeability of the lesion. This can be determine either by monitoring the changes in fluorescein dye leakage over time or by measuring relative leakage of a radiolabelled compound such a [H3] mannitol compared to a reference tissue such as the lung or kidney. This is described in more detail elsewhere (Gehlbach P et al 2003).

An alternative model to the laser rupture is retinal vein occlusion, which reduces overall blood flow to the retina causing a generalised ischemia across the retina.

### REFERENCES

Ace et al, 1989, J Virol 63: 2260-2269

Becker et al., 1998, Hum Gene Ther 9, 1561

Beerrepoot et al., 1996, 56, 3747

Bender et al., 1987, J Virol 61: 1639-1646

Blatt 1994, Anticancer Res., 14, 2109

Blomer et al., 1997, J. Virol. 71: 6641-6649

Bodine et al., 1991, Exp. Hematol. 19: 206-212

Bregni et al., 1998, Gene Ther 5, 465

Burger et al., 1991 J. Gen. Virol. 72. 359-367

Burke et al., 1997, Eur. J. Cancer, 33

Cassel et al., 1993, Exp Haematol 21, 585

Charbord et al 1996 Br J Haematol 94: 449-454

Chou and Roizman, 1992, PNAS 89: 3266-3270

Chou et al., 1994, J. Virol 68: 8304-8311

Clarke and Gordon 1998, J Leukoc Biol 63, 153

Comelisse et al.,1996, Pathol Res Pract 192: 684-693

Cosset et al., 1995, J. Virol. 69: 7430-7436

Cotton et al., 1993 J Virol 67:3777-3785

Crapper et al., 1991, The Lancet, 337, 1304

Dao et al 1997 Blood 89: 446-456

Dunbar et al., 1996, Hum Gene Ther 7:231-253.

Duphar and Emmons, 1994, Stem Cells 12: 563-576

Ema et al., 1997, Proc Natl Acad Sci USA 94, 4273

Emmons et al., 1997, Blood 89, 4040;

Firth et al., 1994, Proc Natl Acad Sci USA 91, 6496

Firth et al., 1995, J Biol Chem 270, 21021

Fisher et al 1996 Virolology 217: 11-22

Flamme et al., 1997, Mech Dev 63, 51

Fraser et al., 1990, Blood 76: 1071-1076

Frey et al., 1998, Blood 91, 2781).

Gorziglia et al., 1996 J. Virol. 70: 4173-4178;

Gosh-Choudhury et al., 1986, Gene 50: 161

Gossen and Bujard, 1992, PNAS 89: 5547-5551

Giardina and Grady 1995, Semin. Hematol. 32, 304

Gossen et al, 1995, Science 268: 1766-1769)

Graham et al., 1977, J Gen Virol 36, 59

Haylock et al., 1992, Blood 80, 1405

Hearing and Shenk 1983, Cell 33, 695

Inoue et al., 1989, J Biol Chem 264: 14954-14959

Jaggar et al., 1997 (Hum Gene Ther 8 2239

Kim et al., 1998, J. Virol., 72, 811-816.

Koong et al., 1994, Cancer Res 54, 1425

Krougliak et al., 1995, Hum. Gen. Ther. 6: 1575

Kuhl et al., 1987, Cell 50, 1057

Levrero et al., 1991, Gene 101: 195

Lieber et al., 1996 J. Virol. 70: 8944-8960

Lonergan et al., 1998, Mol. Cell Biol. 18: 732

MacLean et al., 1991, J Gen Virol 72: 632-639

MacGregor et al., 1991 Methods in Molecular Biology Vol7 ed E. J. Murray p217-235

Madan et al., 1993, Proc. Natl. Acad. Sci. 90: 3928

Malik et al., 1989, Int. J. Cancer 44, 918

Malik et al., 1991, Cancer Res 51, 6643

Maxwell et al., 1999, Nature 399: 271

Meliillo-et al 1996 J. Biol. Chem 272, 12236-12243

Miller, A. D. and G. J. Rosman, 1989, Biotechniques 7(9): p. 980-2, 984-6, 989-90.

Myoshi et al., 1998 J. Virol 72, 8150

Neering et al., 1996 Blood 88, 1147

Payne et al., 1998, J Virol 72, 483.

Pear et al., 1993, Proc Natl Acad Sci 90: 8392-8396

Peshavaria and Day, 1991, Biochem. J. 275: 427-433

Piacibello et al., 1997 Blood 89: 2644-2653

Rice and Knipe, 1990, J. Virol 64: 1704-1715

Santiago-Schwartz et al., 1992, J Leuk Biol 52: 274-281

Semenza and Wang, 1992, Mol. Cell. Biol. 12: 5447-5454

Semenza et al., 1996, J. Biol. Chem 51 32529

Smith et al., 1992, Virology 186: 74-86

Soneoka et al., 1995 Nucl. Acids Res. 23: 628-633

Stevenson et al., 1987, Cancer Res 47, 6100

Stratford-Perricaudet et al., 1992, J. Clin. Invest. 90:626-630

Takenaka et al., 1989, J. Biol. Chem. 264: 2363-2367

Taniguchi et al., 1995, J Cancer Res Clin Oncol 121, 516

Trian et al., 1997, Gene Dev. 11, 72

Voest et al., 1993, Cancer Chemother. Pharmacol. 31, 357

Wang and Semenza, 1993a, PNAS 90: 4304

Wang and Semenza 1993b, Blood, 82, 3610

Wang et al., 1995, J Biol Chem 270, 1230

Ward et al., 1987, Cancer Res 47, 2662

Watanabe et al., 1996, Blood 87, 5032

Watanabe et al., 1998, Leukaemia and Lymphoma 29, 439

Wei et al., 1994, Human Gene Therapy 5, 969

Weisener et al., 1998 Blood 92 2262-2268

Yeh et al., 1996, J. Virol. 70: 559

Zacharova et al., 1997, AIDS Res Hum Retroviruses 13, 719

Zhang et al., 1994, Mol Cell Biol 14, 8085

Having thus described in detail preferred embodiments of the present invention, it is to be understood that the invention defined by the appended claims is not to be limited to particular details set forth in the above description, as many apparent variations thereof are possible without departing from the spirit or scope of the present invention. Modifications and variations of the method and apparatuses described herein will be obvious to those skilled in the art, and are intended to be encompassed by the following claims.

We claim:

1. A method for treating ocular neovascularization comprising delivering to target cells in the eye of a subject in need of treatment, a vector comprising a promoter sequence in operable linkage with a polynucleotide sequence encoding an angiostatic gene product, wherein the angiostatic gene product is expressed in the target cells, thereby treating ocular neovascularization in the subject.

2. The method of claim 1, wherein the promoter sequence is a physiologically regulated promoter sequence or a constitutive promoter sequence.

3. The method of claim 2, wherein the physiologically regulated promoter sequence is a hypoxically responsive promoter sequence.

4. The method of claim 3, wherein the hypoxically responsive promoter sequence is a hypoxic response element (HRE).

5. The method of claim 2, wherein the constitutive promoter sequence is a CMV promoter.

6. The method of claim 1, wherein the ocular neovascularization results in proliferative diabetic retinopathy (PDR) or age-related macular degeneration (AMD) in the subject.

7. The method of claim 1, wherein the ocular neovascularization is choroidal neovascularization or retinal neovascularization.

8. The method of claim 1, wherein the vector is a viral vector.

9. The method of claim 8, wherein the viral vector is a retroviral vector or an adeno-associated viral vector.

10. The method of claim 9, wherein the retroviral vector is a lentiviral vector.

11. The method of claim 10, wherein the lentiviral vector is an EIAV-based lentiviral vector.

12. The method of claim 1, wherein the target cells are retinal cells.

13. The method of claim 12, wherein the retinal cells are retinal pigment epithelial cells.

14. The method of claim 1, wherein delivery of the vector is via direct sub-retinal injection.

15. The method of claim 1, wherein the angiostatic gene product is selected from the group consisting of endostatin, angiostatin, vascular endothelial growth factor receptor 1 (VEGFR1), FLT-1, and pigment epithelium-derived factor (PEDF).

16. The method of claim 1, wherein the vector further comprises a polynucleotide sequence encoding at least one additional angiostatic gene product.

17. The method of claim 16, wherein the at least one additional angiostatic gene product is selected from the group consisting of endostatin, angiostatin, vascular endothelial growth factor receptor 1 (VEGFR1), FLT-1, and pigment epithelium-derived factor (PEDF).

18. The method of claim 1, wherein the angiostatic gene product is endostatin, and wherein the vector further comprises a polynucleotide sequence encoding angiostatin.

19. A vector comprising a hypoxically regulated promoter sequence in operable linkage with a polynucleotide sequence encoding an angiostatic gene product.

20. The vector of claim 19, wherein the vector is a viral vector.

21. The viral vector of claim 20, wherein the viral vector is a retroviral vector or an adeno-associated viral vector.

22. The retroviral vector of claim 21, wherein the retroviral vector is a lentiviral vector.

23. The lentiviral vector of claim 22, wherein the lentiviral vector is an EIAV-based lentiviral vector.

24. The vector of claim 19, wherein the angiostatic gene product is selected from the group consisting of endostatin, angiostatin, vascular endothelial growth factor receptor 1 (VEGFR1), FLT-1, and pigment epithelium-derived factor (PEDF).

25. The vector of claim 19, wherein the vector further comprises a polynucleotide sequence encoding at least one additional angiostatic gene product.

26. The vector of claim 25, wherein the at least one additional angiostatic gene product is selected from the group consisting of endostatin, angiostatin, vascular endothelial growth factor receptor 1 (VEGFR1), FLT-1, and pigment epithelium-derived factor (PEDF).

27. The vector of claim 19, wherein the angiostatic gene product is endostatin, and wherein the vector further comprises a polynucleotide sequence encoding angiostatin.

28. An autoregulatory cassette comprising a polynucleotide comprising:
(i) a construct comprising at least three hypoxia response elements (HRE) operably linked to a promoter;
(ii) a nucleic acid sequence encoding HIF-1, endothelial PAS domain protein (EPAS), or both, operably linked to the construct; and
(iii) one or more nucleic acid sequence(s) of interest (NOI), operably linked to the construct.

29. The polynucleotide of claim 28, wherein the promoter is an SV40 promoter or an MLV promoter.

30. The polynucleotide of claim 28, wherein each of the HREs comprises at least one HIF-1 binding site and wherein each of the HIF-1 binding sites comprises the nucleotide sequence CGTG.

31. The polynucleotide of claim 28, wherein the HREs are direct repeats.

32. The polynucleotide of claim 30, wherein one or more of the HREs comprises a phosphoglycerate kinase (PGK) HRE.

33. The polynucleotide of claim 32, wherein the PGK HRE comprises the nucleic acid sequence of SEQ ID NO:1 or SEQ ID NO:2.

34. The polynucleotide of claim 30, wherein one or more of the HREs comprises erythropoietin (EPO)HRE, LDH HRE, glucose trpt HRE, vascular endothelial cell growth factor (VEGF) HRE, NOS HRE, aldolase HRE, enolase HRE, or heme oxygenase HRE.

35. The polynucleotide of claim 30, wherein one or more of the HREs comprises the nucleic acid sequence of SEQ ID NO:12, 13, 14, 15, 16, 17, 18, 19, 20, 21, 22, or 23.

36. The polynucleotide of claim 28, wherein at least one of the HREs is a mutant HRE having reduced HIF binding activity.

37. The polynucleotide of claim 28, comprising at least four HREs linked to the promoter, wherein at least two of the HREs are positioned upstream (5′) of the promoter and at least two of the HREs are positioned downstream (3′) of the promoter.

38. The polynucleotide of claim 37, comprising at least six HREs, wherein at least three HREs are positioned upstream (5′) of the promoter and at least three HREs are positioned downstream (3′) of the promoter.

39. The polynucleotide of claim 38, wherein at least three of the HREs are phosphoglycerate kinase (PGK) HREs, operably linked to an SV40 promoter or an MLV promoter.

40. A vector comprising the polynucleotide of claim 28.

41. The vector of claim 40, wherein the vector is a viral vector.

42. The viral vector of claim 41, wherein the viral vector further comprises:
(i) a nucleotide sequence encoding an inhibitory RNA molecule capable of affecting the cleavage, directly or indirectly, of VHL RNA;
(ii) one or more inhibitory RNA molecules that binds to and prevents VHL RNA processing, expression, or both; or
(iii) a nucleotide sequence encoding a polypeptide capable of inhibiting the binding of VHL to Elongin B, Elongin C, or both.

43. The viral vector of claim 42, wherein the nucleotide sequence (iii) encodes a non-functional derivative of wild-type VHL.

44. The viral vector of claim 41, wherein the viral vector is a retroviral vector.

45. The viral vector of claim 41, wherein the viral vector is an adenoviral vector.

46. The viral vector of claim 41, wherein the viral vector is a lentiviral vector.


SEQUENCE LISTING (34 sequences)

SEQ ID NO: 1 - DNA, 25 bp, Mus sp.
cgcgtcggtgcaggacgtgacaaat

SEQ ID NO: 2 - DNA, 19 bp, Mus sp.
gtcggtgcaggacgtgaca

SEQ ID NO: 3 - DNA, 243 bp, Artificial Sequence
  Description of Artificial Sequence OBhre1 oligonucleotide
gctagagtcgtgcaggacgtgacatctagtgtcgtgcaggacgtgacatctagtgtcgtgcaggacgtgacagctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctg

SEQ ID NO: 4 - DNA, 229 bp, Artificial Sequence
  Description of Artificial Sequence Synthetic oligonucleotide construct
agctagcctagcgtcgtgcaggacgtgacatctagtgtcgtgcaggacgtgacatctagtgtcgtgcaggacgtgacatctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcgg

SEQ ID NO: 5 - DNA, 225 bp, Artificial Sequence
  Description of Artificial Sequence Synthetic oligonucleotide construct
aagctagctgtcacgtcctgcacgacactagatgtcacgtcctgcacgacactagatgtcacgtcctgcacgactctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcgg

SEQ ID NO: 6 - DNA, 633 bp, Homo sapiens
atggatgccatgaagaggggcctctgctgtgtgctcctgctctgtggggccgtcttcgtcagcccaagcgggaccggctcccacagccaccgcgacttccagcccgtgctgcacctggtggccctgaacagccccctgagcggcggcatgcgcggcatcaggggcgctgacttccagtgcttccagcaggccagggccgtgggcctggccggcaccttccgcgccttcctgagcagccgcctgcaggacctgtacagcatcgtgcgcagggccgaccgcgctgccgtgcccatcgtgaacctgaaggacgagctgctgttccccagctgggaggccctgttcagcggcagcgagggccccctgaagccaggcgccaggatcttcagcttcgacggcaaggacgtgctgcgccaccccacctggccccagaagagcgtgtggcacggctccgaccccaacggccgcaggctgaccgagagctactgcgagacctggcgcaccgaggcccccagcgccaccgggcaggccagctccctgctgggcggcaggctgctgggccagagcgccgccagctgccaccacgcctacatcgtgctgtgcatcgagaacagcttcatgaccgccagcaagtga

SEQ ID NO: 7 - DNA, 633 bp, Homo sapiens
  CDS (1)..(633)
atggatgcaatgaagagagggctctgctgtgtgctgctgctgtgtggagcagtcttcgtttcgcccagcggtaccggatcccacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaagtag

SEQ ID NO: 8 - protein, 210 aa, Homo sapiens
MDAMKRGLCCVLLLCGAVFVSPSGTGSHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 9 - DNA, 912 bp, Homo sapiens
atggatgctatgaagcgcgggctgtgttgcgtcctgctcctgtgcggcgctgtgtttgtgtccccctccggcaccgggagcctgttcgagaagaaggtgtacctgagcgagtgcaagaccggcaacggcaagaactacaggggcaccatgagcaagaccaagaacggcatcacctgccagaagtggagcagcaccagcccccacaggcctcgcttcagccccgccacccaccccagcgagggcctggaggagaactactgccgcaaccccgacaacgacccccagggcccttggtgctacaccaccgaccctgagaagcgctacgactactgcgacatcctggagtgcgaggaagagtgtatgcactgcagcggggagaactacgacggcaagatcagcaagaccatgagcggcctggagtgccaggcctgggactcccagagcccccacgcccacggctacatccccagcaagttccccaacaagaacctgaagaagaactattgtcgcaatcccgaccgcgagctgcgcccctggtgcttcaccaccgatcccaacaagcgctgggagctgtgcgacatcccccgctgcaccacccccccacccagcagcggccccacctaccagtgcctgaagggcaccggcgagaattaccgcggcaacgtggccgtgaccgtgagcggccacacctgccagcactggagcgcccagaccccccacacccacaaccgcacccccgagaacttcccctgcaagaacctcgacgagaattattgccggaaccctgacggcaagagggccccctggtgccacaccaccaacagccaggtgcgctgggagtactgcaagatccccagctgcgacagcagccccgtgagcaccgagcagctggccccaaccgcccctccctga

SEQ ID NO: 10 - DNA, 912 bp, Homo sapiens
  CDS (1)..(912)
atggatgcaatgaagagagggctctgctgtgtgctgctgctgtgtggagcagtcttcgtttcgcccagcggtaccggatccttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaactttccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccaccttaa

SEQ ID NO: 11 - protein, 303 aa, Homo sapiens
MDAMKRGLCCVLLLCGAVFVSPSGTGSLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPP

SEQ ID NO: 12 - DNA, 12 bp, Artificial Sequence
  Description of Artificial Sequence spacer oligonucleotide
gtcgtgcaggca

SEQ ID NO: 13 - DNA, 24 bp, Artificial Sequence
  Description of Artificial Sequence spacer oligonucleotide
tctagtgtcgtgcaggcatctagt

SEQ ID NO: 14 - DNA, 25 bp, Artificial Sequence
  Description of Artificial Sequence spacer oligonucleotide
cgcgtcggtgcaggacgtgacaaat

SEQ ID NO: 15 - DNA, 19 bp, Artificial Sequence
  Description of Artificial Sequence spacer oligonucleotide
gtcggtgcaggacgtgaca

SEQ ID NO: 16 - DNA, 18 bp, Mus sp.
gtcgtgcagtacgtgaca

SEQ ID NO: 17 - DNA, 26 bp, Mus sp.
gggccctacgtgctgtctcacacagc

SEQ ID NO: 18 - DNA, 26 bp, Mus sp.
gggccctacgtgctgcctcgcatggc

SEQ ID NO: 19 - DNA, 24 bp, Mus sp.
cgcgtcgtgcaggacgtgacaaat

SEQ ID NO: 20 - DNA, 30 bp, Mus sp.
ccagcggacgtgcgggaacccacgtgtagg

SEQ ID NO: 21 - DNA, 26 bp, Homo sapiens
tccacaggcgtgccgtctgacacgca

SEQ ID NO: 22 - DNA, 35 bp, Homo sapiens
ccacagtgcatacgtgggctccaacaggtcctctt

SEQ ID NO: 23 - DNA, 24 bp, Rattus sp.
acagtgcatacgtgggcttccaca

SEQ ID NO: 24 - DNA, 17 bp, Homo sapiens
actacgtgctgcctagg

SEQ ID NO: 25 - DNA, 26 bp, Homo sapiens
cccctcggacgtgactcggaccacat

SEQ ID NO: 26 - DNA, 37 bp, Homo sapiens
acgctgagtgcgtgcgggactcggagtacgtgacgga

SEQ ID NO: 27 - DNA, 28 bp, Homo sapiens
cggacgtgctggcgtggcacgtcctctc

SEQ ID NO: 28 - DNA, 249 bp, Artificial Sequence
  Description of Artificial Sequence XiaMac promoter
gctagagtcgtgcaggacgtgacatctagtgtcgtgcaggacgtgacatctagtgtcgtgcaggacgtgacagctagcattccatcacgtggcccgagagaagcatccggagtactacaaggactgctgacagcgagatttctacaagggactttccgctggggactttccagggaggtgtggcctgggcgggactggggagtggcgagccctcagatgctgcatataagcagcagctgcttttgcccc

SEQ ID NO: 29 - DNA, 273 bp, Artificial Sequence
  Description of Artificial Sequence IRF-1 responsive nucleotide sequence
gctagagtcgtgcaggacgtgacatctagtgtcgtgcaggacgtgacatctagtgtcgtgcaggacgtgacagctagcattccatcacgtggcccgagagaagcatccggagtactacaaggactgctgacagcgagatttctacaagggactttccgctggggactttccagggaggtgtggcctgggcgggactggggagtggcaagtgaaagtgaaagtgaaagtgagagccctcagatgctgcatataagcagcagctgcttttgcccc

SEQ ID NO: 30 - DNA, 243 bp, Artificial Sequence; identical to SEQ ID NO: 3
  Description of Artificial Sequence Synthetic promoter construct

SEQ ID NO: 31 - DNA, 18 bp, Artificial Sequence
  Description of Artificial Sequence Synthetic oligonucleotide
tgagttaattaaggatcc

SEQ ID NO: 32 - DNA, 21 bp, Artificial Sequence
  Description of Artificial Sequence Synthetic oligonucleotide
ctcgagtcgcgagccaccatg

SEQ ID NO: 33 - protein, 6 aa, Artificial Sequence
  Description of Artificial Sequence 6-His tag
HHHHHH

SEQ ID NO: 34 - DNA, 19 bp, Artificial Sequence
  Description of Artificial Sequence Synthetic oligonucleotide
tcgagctagcgtcgactcg